(19) United States
(12) Patent Application Publication
(10) Pub. No.: US 2021/0106581 A1
(43) Pub. Date: Apr. 15, 2021

(54) Treatment Of Liver Diseases

(71) Applicant: Melior Pharmaceuticals I, Inc., Exton, PA (US)

(72) Inventors: Andrew G. Reaume, Exton, PA (US); Wei-Na Cong, Exton, PA (US)

(21) Appl. No.: 16/970,543

(22) PCT Filed: Feb. 19, 2019
(86) PCT No.: PCT/US19/18484

§ 371 Date: Aug. 17, 2020

Related U.S. Application Data
(60) Provisional application No. 62/633,249, filed on Feb. 21, 2018.
(60) Provisional application No. 62/743,272, filed on Oct. 9, 2018.

(51) Int. Cl. A61K 31/513; A61P 1/16

(52) U.S. Cl. CPC A61K 31/513; A61K 45/06; A61P 1/16

(57) ABSTRACT

Methods of treating non-alcoholic steatohepatisis (NASH), non-alcoholic fatty liver disease (NAFLD), fatty acid liver disease (FALD), alcoholic liver disease, and/or liver fibrosis in a mammal by administering a lyn kinase activator are provided herein.

### FIELD

The present disclosure is directed, in part, to methods of treating liver diseases, such as non-alcoholic steatohepatisis (NASH), non-alcoholic fatty liver disease (NAFLD), fatty acid liver disease (FALD), alcoholic liver disease, and/or liver fibrosis, by administering a lyn kinase activator.

### BACKGROUND

Accumulation of lipids in the liver, either due to excessive intake or inability to eliminate lipids, occurs in deposits which, leads to transformation of hepatic cellular structure which is referred to as steatosis. Over time, a persistent condition of hepatic steatosis can lead to liver damage and hepatic fibrosis with a constellation of pathological consequences. A key marker of hepatic steatosis is hepatic triglyceride accumulation. Non-alcoholic steatohepatitis (NASH) is liver inflammation and damage caused by a buildup of fat in the liver. It is part of a group of conditions called non-alcoholic fatty liver disease (NAFLD). In some people, the fat causes inflammation and damages cells in the liver. NASH can get worse and cause scarring of the liver, which leads to liver fibrosis and/or cirrhosis.

Lyn kinase is a member of the src family of non-receptor protein tyrosine kinases that is predominantly expressed in B-lymphoid and myeloid cells (Briggs et al., Biochemistry, 2000, 39, 489-495). Lyn kinase participates in signal transduction from cell surface receptors that lack intrinsic tyrosine kinase activity. Activation of the lyn kinase activity is necessary for proliferation of CD45⁺ myeloma cells stimulated by IL-6 (Ishikawa et al, Blood, 2002, 99, 2172-2178). Association of lyn and fyn with the proline-rich domain of glycoprotein VI regulates intracellular signaling (Suzuki-Inoue et al., J. Biol. Chem., 2002, 277, 21561-21566). The lyn/CD22/SHP-1 pathway is also important in autoimmunity (Blasioli et al., Curr. Dir. Autoimmun., 2002, 5, 151-160).

### SUMMARY

The present disclosure provides methods of treating NASH, NAFLD, FALD, alcoholic liver disease, and/or liver fibrosis in a mammal in need thereof, comprising administering to the mammal a compound having the formula:

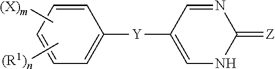

wherein: R¹ is an alkyl group; X is a halogen; Y is O, S, or NH; Z is O or S; and n is an integer from 0 to 5 and m is 0 or 1, wherein m+n is less than or equal to 5; or a pharmaceutically acceptable salt thereof.

The present disclosure also provides methods of treating NASH, NAFLD, FALD, alcoholic liver disease, and/or liver fibrosis in a mammal in need thereof, comprising administering to the mammal a compound having the formula:

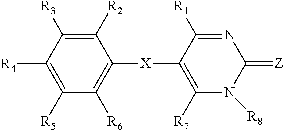

wherein: each of R₁, R₂, R₃, R₄, R₅, R₆, and R₇ is, independently, a hydrogen, alkoxy, alkyl, alkenyl, alkynyl, aryl, aryloxy, benzyl, cycloalkyl, halogen, heteroaryl, heterocycloalkyl, —CN, —OH, —NO₂, —CF₃, —CO₂H, —CO₂alkyl, or —NH₂; R₈ is an alkyl or hydrogen; X is O, S, NH, or N-alkyl; and Z is O or S; or a pharmaceutically acceptable salt thereof.

The present disclosure also provides methods of treating NASH, NAFLD, FALD, alcoholic liver disease, and/or liver fibrosis in a mammal in need thereof, comprising administering to the mammal a compound having the formula:

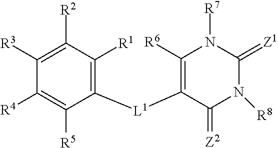

wherein:

each of R¹, R², R³, R⁴, and R⁵ is, independently, H, halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, NR^{c1}S(O)₂NR^{c1}R^{d1}, S(O)R^{b1}, S(O)NR^{c1}R^{d1}, S(O)₂R^{b1}, NR^{c1}S(O)₂R^{b1}, or S(O)₂NR^{c1}R^{d1}, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4, or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2} NR^{c2}S(O)₂R^{b2}, and S(O)₂NR^{c2}R^{d2}; or two adjacent groups of R¹, R², R³, R⁴, and R⁵ can link to form a fused cycloalkyl or fused heterocycloalkyl group, each optionally substituted by 1, 2, or 3 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2}; R⁶ is H, halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, NR^{c1}S(O)₂NR^{c1}R^{d1}, S(O)R^{b1}, S(O)NR^{c1}R^{d1}, S(O)₂R^{b1}, NR^{c1}S(O)₂R^{b1}, or S(O)₂NR^{c1}R^{d1}, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4, or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2}; R⁷ is H, C₁₋₆alkyl, C₁₋₆haloalkyl, C₁₋₆hydroxyalkyl, C(O)R^{b1}, C(O)NR^{c1}R^{d1}, C(O)OR^{a1}, S(O)R^{b1}, S(O)NR^{c1}R^{d1}, S(O)₂R^{b1}, or S(O)₂NR^{c1}R^{d1}; R⁸ is H, C₁₋₆alkyl, C₁₋₆haloalkyl, C₁₋₆hydroxyalkyl, C(O)R^{b1}, C(O)NR^{c1}R^{d1}, C(O)OR^{a1}, S(O)R^{b1}, S(O)NR^{c1}R^{d1}, S(O)₂R^{b1}, or S(O)₂NR^{c1}R^{d1}; R^{a1}, R^{b1}, R^{c1}, and R^{d1} are each, independently, selected from H, C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, and heterocycloalkylalkyl, wherein each of C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, and heterocycloalkylalkyl is optionally substituted with 1, 2, 3, 4, or 5 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy; or R^{c1} and R^{d1} together with the N atom to which they are attached form a 4-, 5-, 6-, or 7-membered heterocycloalkyl group or heteroaryl group, each optionally substituted with 1, 2, or 3 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy; R^{a2}, R^{b2}, R^{c2}, and R^{d2} are each, independently, selected from H, C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, and heterocycloalkylalkyl, wherein each of C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, or heterocycloalkylalkyl is optionally substituted with 1, 2, or 3 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy; or R^{c2} and R^{d2} together with the N atom to which they are attached form a 4-, 5-, 6-, or 7-membered heterocycloalkyl group or heteroaryl group, each optionally substituted with 1, 2, or 3 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy; Z¹ is O, S, or NR⁹; R⁹ is H, OH, C₁₋₆alkoxy, C₁₋₆haloalkyl, C₁₋₆hydroxyalkyl, aryloxy, heteroaryloxy, CN, or NO₂; Z² is O, S, or NR¹⁰; R¹⁰ is H, OH, C₁₋₆alkoxy, C₁₋₆haloalkyl, C₁₋₆hydroxyalkyl, aryloxy, heteroaryloxy, CN, or NO₂; L¹ is O, S, or NR¹¹; and R¹¹ is H, C₁₋₆alkyl, C₁₋₆haloalkyl, C₁₋₆hydroxyalkyl, C(O)R^{b1}, C(O)NR^{c1}R^{d1}, C(O)OR^{a1}, S(O)R^{b1}, S(O)NR^{c1}R^{d1}, S(O)₂R^{b1}, or S(O)₂NR^{c1}R^{d1}; or a pharmaceutically acceptable salt thereof.

The present disclosure also provides methods of treating NASH, NAFLD, FALD, alcoholic liver disease, and/or liver fibrosis in a mammal in need thereof, comprising administering to the mammal a compound having the formula:

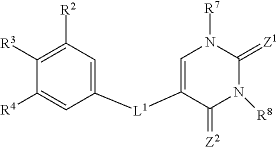

wherein: R², R³, and R⁴ are each, independently, H, halo, C₁₋₆alkyl, C₁₋₆hydroxyalkyl, or C₁₋₆haloalkyl; R⁷ is H, C₁₋₆alkyl, C(O)R^{b1}, C(O)NR^{c1}R^{d1}, or C(O)OR^{a1}; R⁸ is H, C₁₋₆alkyl, C(O)R^{b1}, C(O)NR^{c1}R^{d1}, or C(O)OR^{a1}; R^{a1}, R^{b1}, R^{c1}, and R^{d1} are each, independently, selected from H, C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, and heterocycloalkylalkyl, wherein each of C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, and heterocycloalkylalkyl is optionally substituted with 1, 2, 3, 4, or 5 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy; or R^{c1} and R^{d1} together with the N atom to which they are attached form a 4-, 5-, 6-, or 7-membered heterocycloalkyl group or heteroaryl group, each optionally substituted with 1, 2, or 3 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy; Z¹ is O or S; Z² is O or S; and L is O or S; or a pharmaceutically acceptable salt thereof.

The present disclosure also provides methods of treating NASH, NAFLD, FALD, alcoholic liver disease, and/or liver fibrosis in a mammal in need thereof, comprising administering to the mammal a compound having the formula:

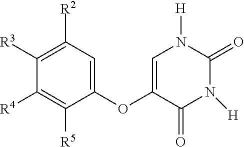

wherein: R², R³, R⁴, and R⁵ are each, independently, H, F, Cl, CH₃, SCH₃, OCH₃, C(CH₃)₃, CH(CH₃)₂, or C₂H₅; or a pharmaceutically acceptable salt thereof.

The present disclosure also provides methods of treating NASH, NAFLD, FALD, alcoholic liver disease, and/or liver fibrosis in a mammal in need thereof, comprising administering to the mammal a compound having the formula:

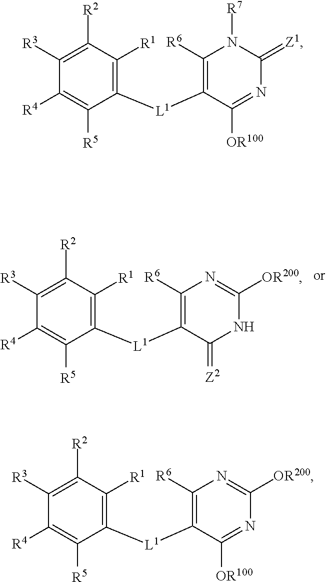

wherein:

each of R¹, R², R³, R⁴, and R⁵ is, independently, H, halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, NR^{c1}S(O)₂NR^{c1}R^{d1}, S(O)R^{b1}, S(O)NR^{c1}R^{d1}, S(O)₂R^{b1}, NR^{c1}S(O)₂R^{b1}, or S(O)₂NR^{c1}R^{d1}, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4, or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2}; or two adjacent groups of R¹, R², R³, R⁴, and R⁵ can link to form a fused cycloalkyl or fused heterocycloalkyl group, each optionally substituted by 1, 2, or 3 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2}; R⁶ is H, halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, NR^{c1}S(O)₂NR^{c1}R^{d1}, S(O)R^{b1}, S(O)NR^{c1}R^{d1}, S(O)₂R^{b1}, NR^{c1}S(O)₂R^{b1}, or S(O)₂NR^{c1}R^{d1}, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4, or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2}; R is H, C₁₋₆alkyl, C₁₋₆haloalkyl, C(O)R^{b1}, C(O)NR^{c1}R^{d1}, C(O)OR^{a1}, S(O)R^{b1}, S(O)NR^{c1}R^{d1}, S(O)₂R^{b1}, or S(O)₂NR^{c1}R^{d1}; R^{a1}, R^{b1}, R^{c1}, and R^{d1} are each, independently, selected from H, C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, and heterocycloalkylalkyl, wherein each of C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, and heterocycloalkylalkyl is optionally substituted with 1, 2, 3, 4, or 5 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy; or R^{c1} and R^{d1} together with the N atom to which they are attached form a 4-, 5-, 6-, or 7-membered heterocycloalkyl group or heteroaryl group, each optionally substituted with 1, 2, or 3 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy; R^{a2}, R^{b2}, R^{c2}, and R^{d2} are each, independently, selected from H, C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, and heterocycloalkylalkyl, wherein each of C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, or heterocycloalkylalkyl is optionally substituted with 1, 2, or 3 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy; or R^{c2} and R^{d2} together with the N atom to which they are attached form a 4-, 5-, 6-, or 7-membered heterocycloalkyl group or heteroaryl group, each optionally substituted with 1, 2, or 3 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy; Z¹ is O, S, or NR⁹; R⁹ is H, OH, C₁₋₆alkoxy, C₁₋₆haloalkyl, C₁₋₆hydroxyalkyl, aryloxy, heteroaryloxy, CN, or NO₂; Z² is O, S, or NR¹⁰; R¹⁰ is H, OH, C₁₋₆alkoxy, C₁₋₆haloalkyl, C₁₋₆hydroxyalkyl, aryloxy, heteroaryloxy, CN, or NO₂; L¹ is O, S, or NR¹¹; R¹¹ is H, C₁₋₆alkyl, C₁₋₆haloalkyl, C₁₋₆hydroxyalkyl, C(O)R^{b1}, C(O)NR^{c1}R^{d1}, C(O)OR^{a1}, S(O)R^{b1}, S(O)NR^{c1}R^{d1}, S(O)₂R^{b1}, or S(O)₂NR^{c1}R^{d1}; R¹⁰⁰ is a hydroxyl protecting group, C₁₋₆alkyl, C₂₋₆alkenyl, C₂₋₆alkynyl, C₁₋₆haloalkyl, C₁₋₆hydroxyalkyl, C₃₋₆cycloalkyl, heterocycloalkyl, aryl, heteroaryl, C(O)R^{b1}, C(O)NR^{c1}R^{d1}, C(O)OR^{a1}, S(O)R^{b1}, S(O)NR^{c1}R^{d1}, S(O)₂R^{b1}, S(O)₂NR^{c1}R^{d1}, S(O)₂OR^{e1}, P(O)OR^{f1}OR^{g1}, or Si(R^{h1})₃, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4 or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{e2}R^{f2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2}; R²⁰⁰ is a hydroxyl protecting group, C₁₋₆alkyl, C₂₋₆alkenyl, C₂₋₆alkynyl, C₁₋₆haloalkyl, C₁₋₆hydroxyalkyl, C₃₋₆cycloalkyl, heterocycloalkyl, aryl, heteroaryl, C(O)R^{b1}, C(O)NR^{c1}R^{d1}, C(O)OR^{a1}, S(O)R^{b1}, S(O)NR^{c1}R^{d1}, S(O)₂R^{b1}, S(O)₂NR^{c1}R^{d1}, S(O)₂OR^{e1}, P(O)OR^{f1}OR^{g1}, or Si(R^{h1})₃, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4 or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2}; each R^{e1} is, independently, H, C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, cycloalkylalkyl, heterocycloalkylalkyl, arylalkyl, or heteroarylalkyl; each R^{f1} is, independently, H, C₁₋₆alkyl, C₁₋₆haloalkyl, C₁₋₆hydroxyalkyl, C₂₋₆alkenyl, (C₁₋₆alkoxy)-C₁₋₆alkyl, C₂₋₆alkynyl, aryl, cycloalkyl, heteroaryl, heterocycloalkyl, arylalkyl, cycloalkylalkyl, heteroarylalkyl, or heterocycloalkylalkyl; each R^{g1} is, independently, H, C₁₋₆alkyl, C₁₋₆haloalkyl, C₁₋₆hydroxyalkyl, C₂₋₆alkenyl, C₂₋₆alkynyl, aryl, cycloalkyl, heteroaryl, or heterocycloalkyl; and each R^{h1} is, independently, H, C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, cycloalkylalkyl, heterocycloalkylalkyl, arylalkyl, or heteroarylalkyl; or a pharmaceutically acceptable salt thereof.

### BRIEF DESCRIPTION OF THE DRAWINGS

FIG. 1 shows that MLR-1023 significantly reduced circulating serum triglyceride levels in animals on a high fat diet, and otherwise prone to exhibiting elevated triglycerides compared to animals on a normal diet.

FIG. 2 shows that MLR-1023 significantly reduced the accumulation of triglycerides in the liver on animals on a high fat diet and otherwise prone to accumulating elevated triglycerides compared to animals on a normal diet.

FIG. 3 shows that body weight is significantly reduced due to treatment with MLR-1023.

FIG. 4 shows that liver weight is significantly reduced due to treatment with MLR-1023.

FIG. 5 shows that that fasting serum total cholesterol is significantly reduced due to treatment with MLR-1023.

FIG. 6 shows that NAFLD activity score (NAS) score is significantly reduced due to the 30 mg/kg MLR-1023 treatment.

FIG. 7 shows that hepatocellular ballooning is significantly reduced due to the 100 mpk MLR-1023 treatment.

FIGS. 8A and 8B show the histological evidence of reduced steatosis and hepatocellular ballooning responsible for the reduced NAS score with MLR-1023 treatment.

FIG. 9 shows terminal blood liver enzyme changes due to MLR-1023 treatment.

FIG. 10 shows hydroxyproline content changes due to MLR-1023 treatment.

FIG. 11 shows hepatic histological changes due to MLR-1023 treatment.

FIG. 12 shows histological scoring of inflammation and mineralization/necrosis upon treatment with MLR-1023.

### DESCRIPTION OF EMBODIMENTS

As used herein, the terms “a” or “an” means that “at least one” or “one or more” unless the context clearly indicates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by 10% and remain within the scope of the disclosed embodiments.

As used herein, the term “alkoxy” means a straight or branched —O-alkyl group of 1 to 20 carbon atoms, including, but not limited to, methoxy, ethoxy, n-propoxy, isopropoxy, t-butoxy, and the like. In some embodiments, the alkoxy chain is from 1 to 10 carbon atoms in length, from 1 to 8 carbon atoms in length, from 1 to 6 carbon atoms in length, from 1 to 4 carbon atoms in length, from 2 to 10 carbon atoms in length, from 2 to 8 carbon atoms in length, from 2 to 6 carbon atoms in length, or from 2 to 4 carbon atoms in length. An alkoxy group can be unsubstituted or substituted with one or two suitable substituents.

As used herein, the term “alkyl” means a saturated hydrocarbon group which is straight-chained or branched. An alkyl group can contain from 1 to 20, from 2 to 20, from 1 to 10, from 2 to 10, from 1 to 8, from 2 to 8, from 1 to 6, from 2 to 6, from 1 to 4, from 2 to 4, from 1 to 3, or 2 or 3 carbon atoms. Examples of alkyl groups include, but are not limited to, methyl (Me), ethyl (Et), propyl (e.g., n-propyl and isopropyl), butyl (e.g., n-butyl, t-butyl, isobutyl), pentyl (e.g., n-pentyl, isopentyl, neopentyl), hexyl, isohexyl, heptyl, 4,4-dimethylpentyl, octyl, nonyl, decyl, 2,2,4-trimethylpentyl, undecyl, dodecyl, 2-methyl-1-propyl, 2-methyl-2-propyl, 2-methyl-1-butyl, 3-methyl-1-butyl, 2-methyl-3-butyl, 2-methyl-1-pentyl, 2,2-dimethyl-1-propyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, and the like. An alkyl group can be unsubstituted or substituted with one or two suitable substituents.

As used herein, the term “alkenyl” means a straight or branched alkyl group having one or more double carbon-carbon bonds and 2-20 carbon atoms, including, but not limited to, ethenyl, 1-propenyl, 2-propenyl, 2-methyl-1-propenyl, 1-butenyl, 2-butenyl, vinyl, allyl, pentenyl, hexenyl, butadienyl, pentadienyl, hexadienyl, 2-ethylhexenyl, 2-propyl-2-butenyl, 4-(2-methyl-3-butene)-pentenyl and the like. In some embodiments, the alkenyl chain is from 2 to 10 carbon atoms in length, from 2 to 8 carbon atoms in length, from 2 to 6 carbon atoms in length, or from 2 to 4 carbon atoms in length. The double bond of an alkenyl group can be unconjugated or conjugated to another unsaturated group. An alkenyl group can be unsubstituted or substituted with one or two suitable substituents.

As used herein, the term “alkynyl” means a straight or branched alkyl group having one or more triple carbon-carbon bonds and 2-20 carbon atoms, including, but not limited to, acetylene, 1-propylene, 2-propylene, ethynyl, propynyl, butynyl, pentynyl, hexynyl, methylpropynyl, 4-methyl-1-butynyl, 4-propyl-2-pentynyl, and 4-butyl-2-hexynyl, and the like. In some embodiments, the alkynyl chain is 2 to 10 carbon atoms in length, from 2 to 8 carbon atoms in length, from 2 to 6 carbon atoms in length, or from 2 to 4 carbon atoms in length. The triple bond of an alkynyl group can be unconjugated or conjugated to another unsaturated group. An alkynyl group can be unsubstituted or substituted with one or two suitable substituents.

As used herein, the term “animal” includes, but is not limited to, humans and non-human vertebrates such as wild, domestic, and farm animals.

As used herein, the term “aryl” means a monocyclic, bicyclic, or polycyclic (e.g., having 2, 3 or 4 fused rings) aromatic hydrocarbons. In some embodiments, aryl groups have from 6 to 20 carbon atoms or from 6 to 10 carbon atoms. Examples of aryl groups include, but are not limited to, phenyl, naphthyl, anthracenyl, phenanthrenyl, indanyl, indenyl, tolyl, fluorenyl, tetrahydronaphthyl, azulenyl, naphthyl, 5,6,7,8-tetrahydronaphthyl, and the like. An aryl group can be unsubstituted or substituted with one or two suitable substituents.

As used herein, the term “aryloxy” means an —O-aryl group, wherein aryl is as defined herein. An aryloxy group can be unsubstituted or substituted with one or two suitable substituents. The aryl ring of an aryloxy group can be a monocyclic ring, wherein the ring comprises 6 carbon atoms, referred to herein as “(CG)aryloxy.”

As used herein, the term “benzyl” means —CH₂-phenyl.

As used herein, the term “carbonyl” group is a divalent group of the formula —C(O)—.

As used herein, the term “carrier” means a diluent, adjuvant, or excipient with which a compound is administered. Pharmaceutical carriers can be liquids, such as water and oils, including those of petroleum, animal, vegetable or synthetic origin, such as peanut oil, soybean oil, mineral oil, sesame oil and the like. The pharmaceutical carriers can also be saline, gum acacia, gelatin, starch paste, talc, keratin, colloidal silica, urea, and the like. In addition, auxiliary, stabilizing, thickening, lubricating and coloring agents can be used.

As used herein, the term, “compound” means all stereoisomers, tautomers, and isotopes of the compounds described herein.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

As used herein, the term “cycloalkyl” means non-aromatic cyclic hydrocarbons including cyclized alkyl, alkenyl, and alkynyl groups that contain up to 20 ring-forming carbon atoms. Cycloalkyl groups can include mono- or polycyclic ring systems such as fused ring systems, bridged ring systems, and spiro ring systems. In some embodiments, polycyclic ring systems include 2, 3, or 4 fused rings. A cycloalkyl group can contain from 3 to 15, from 3 to 10, from 3 to 8, from 3 to 6, from 4 to 6, from 3 to 5, or 5 or 6 ring-forming carbon atoms. Ring-forming carbon atoms of a cycloalkyl group can be optionally substituted by oxo or sulfido. Examples of cycloalkyl groups include, but are not limited to, cyclopropyl, cyclobutyl, cyclopentyl, cyclohexyl, cycloheptyl, cyclooctyl, cyclononyl, cyclopentenyl, cyclohexenyl, cyclohexadienyl, cycloheptatrienyl, norbomyl, norpinyl, norcarnyl, adamantyl, and the like. Also included in the definition of cycloalkyl are moieties that have one or more aromatic rings fused (having a bond in common with) to the cycloalkyl ring, for example, benzo or thienyl derivatives of pentane, pentene, hexane, and the like (e.g., 2,3-dihydro-1H-indene-1-yl or 1H-inden-2(3H)-one-1-yl). A cycloalkyl group can be unsubstituted or substituted by one or two suitable substituents.

As used herein, the term “halogen” means fluorine, chlorine, bromine, or iodine. Correspondingly, the meaning of the terms “halo” and “Hal” encompass fluoro, chloro, bromo, and iodo.

As used herein, the term “heteroaryl” means an aromatic heterocycle having up to 20 ring-forming atoms (e.g., C) and having at least one heteroatom ring member (ring-forming atom) such as sulfur, oxygen, or nitrogen. In some embodiments, the heteroaryl group has at least one or more heteroatom ring-forming atoms, each of which are, independently, sulfur, oxygen, or nitrogen. In some embodiments, the heteroaryl group has from 3 to 20 ring-forming atoms, from 3 to 10 ring-forming atoms, from 3 to 6 ring-forming atoms, or from 3 to 5 ring-forming atoms. In some embodiments, the heteroaryl group contains 2 to 14 carbon atoms, from 2 to 7 carbon atoms, 2 to 5 carbon atoms, or 5 or 6 carbon atoms. In some embodiments, the heteroaryl group has 1 to 4 heteroatoms, 1 to 3 heteroatoms, or 1 or 2 heteroatoms. Heteroaryl groups include monocyclic and polycyclic (e.g., having 2, 3 or 4 fused rings) systems. Examples of heteroaryl groups include, but are not limited to, pyridyl, pyrimidinyl, pyrazinyl, pyridazinyl, triazinyl, furyl, quinolyl, isoquinolyl, thienyl, imidazolyl, thiazolyl, indolyl (such as indol-3-yl), pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, pyranyl, oxadiazolyl, isoxazolyl, triazolyl, thianthrenyl, pyrazolyl, indolizinyl, isoindolyl, isobenzofuranyl, benzoxazolyl, xanthenyl, 2H-pyrrolyl, pyrrolyl, 3H-indolyl, 4H-quinolizinyl, phthalazinyl, naphthyridinyl, quinazolinyl, phenanthridinyl, acridinyl, perimidinyl, phenanthrolinyl, phenazinyl, isothiazolyl, phenothiazinyl, isoxazolyl, furazanyl, phenoxazinyl, pyrazyl, phienyl, groups, and the like. Suitable heteroaryl groups include 1,2,3-triazole, 1,2,4-triazole, 5-amino-1,2,4-triazole, imidazole, oxazole, isoxazole, 1,2,3-oxadiazole, 1,2,4-oxadiazole, 3-amino-1,2,4-oxadiazole, 1,2,5-oxadiazole, 1,3,4-oxadiazole, pyridine, and 2-aminopyridine. A heteroaryl group can be unsubstituted or substituted with one or two suitable substituents.

As used herein, the term “heterocycle” or “heterocyclic ring” means a 5- to 7-membered mono- or bicyclic or 7- to 10-membered bicyclic heterocyclic ring system any ring of which may be saturated or unsaturated, and which consists of carbon atoms and from one to three heteroatoms chosen from N, O and S, and wherein the N and S heteroatoms may optionally be oxidized, and the N heteroatom may optionally be quaternized, and including any bicyclic group in which any of the above-defined heterocyclic rings is fused to a benzene ring. Particularly useful are rings containing one oxygen or sulfur, one to three nitrogen atoms, or one oxygen or sulfur combined with one or two nitrogen atoms. The heterocyclic ring may be attached at any heteroatom or carbon atom which results in the creation of a stable structure. Examples of heterocyclic groups include, but are not limited to, piperidinyl, piperazinyl, 2-oxopiperazinyl, 2-oxopiperidinyl, 2-oxopyrrolodinyl, 2-oxoazepinyl, azepinyl, pyrrolyl, 4-piperidonyl, pyrrolidinyl, pyrazolyl, pyrazolidinyl, imidazolyl, imidazolinyl, imidazolidinyl, pyridyl, pyrazinyl, pyrimidinyl, pyridazinyl, oxazolyl, oxazolidinyl, isoxazolyl, isoxazolidinyl, morpholinyl, thiazolyl, thiazolidinyl, isothiazolyl, quinuclidinyl, isothiazolidinyl, indolyl, quinolinyl, isoquinolinyl, benzimidazolyl, thiadiazoyl, benzopyranyl, benzothiazolyl, benzoxazolyl, furyl, tetrahydrofuryl, tetrahydropyranyl, thienyl, benzothienyl, thiamorpholinyl, thiamorpholinyl sulfoxide, thiamorpholinyl sulfone, and oxadiazolyl. Morpholino is the same as morpholinyl.

As used herein, the term “heterocycloalkyl” means non-aromatic heterocycles having up to 20 ring-forming atoms including cyclized alkyl, alkenyl, and alkynyl groups, where one or more of the ring-forming carbon atoms is replaced by a heteroatom such as an O, N, or S atom. Hetercycloalkyl groups can be mono or polycyclic (e.g., fused, bridged, or spiro systems). In some embodiments, the heterocycloalkyl group has from 1 to 20 carbon atoms, or from 3 to 20 carbon atoms. In some embodiments, the heterocycloalkyl group contains 3 to 14 ring-forming atoms, 3 to 7 ring-forming atoms, or 5 or 6 ring-forming atoms. In some embodiments, the heterocycloalkyl group has 1 to 4 heteroatoms, 1 to 3 heteroatoms, or 1 or 2 heteroatoms. In some embodiments, the heterocycloalkyl group contains 0 to 3 double bonds. In some embodiments, the heterocycloalkyl group contains 0 to 2 triple bonds. Examples of heterocycloalkyl groups include, but are not limited to, morpholino, thiomorpholino, piperazinyl, tetrahydrofuranyl, tetrahydrothienyl, 2,3-dihydrobenzofuryl, 1,3-benzodioxole, piperidinyl, benzo-1,4-dioxane, pyrrolidinyl, isoxazolidinyl, oxazolidinyl, isothiazolidinyl, pyrazolidinyl, thiazolidinyl, imidazolidinyl, pyrrolidino, piperidino, morpholinyl, thiomorpholinyl, pyranyl, pyrrolidin-2-one-3-yl, and the like. In addition, ring-forming carbon atoms and heteroatoms of a heterocycloalkyl group can be optionally substituted by oxo or sulfido. For example, a ring-forming S atom can be substituted by 1 or 2 oxo (form a S(O) or S(O)₂). For another example, a ring-forming C atom can be substituted by oxo (form carbonyl). Also included in the definition of heterocycloalkyl are moieties that have one or more aromatic rings fused (having a bond in common with) to the nonaromatic heterocyclic ring including, but not limited to, pyridinyl, thiophenyl, phthalimidyl, naphthalimidyl, and benzo derivatives of heterocycles such as indolene, isoindolene, isoindolin-1-one-3-yl, 4,5,6,7-tetrahydrothieno[2,3-c]pyridine-5-yl, 5,6-dihydrothieno[2,3-c]pyridin-7(4H)-one-5-yl, and 3,4-dihydroisoquinolin-1(2H)-one-3yl groups. Ring-forming carbon atoms and heteroatoms of the heterocycloalkyl group can be optionally substituted by oxo or sulfido. A heterocycloalkyl group can be unsubstituted or substituted with one or two suitable substituents.

As used herein, the term “individual” or “patient,” used interchangeably, means any animal, including mammals, such as mice, rats, other rodents, rabbits, dogs, cats, swine, cattle, sheep, horses, or primates, such as humans.

As used herein, the phrase “in need thereof” means that the animal or mammal has been identified as having a need for the particular method or treatment. In some embodiments, the identification can be by any means of diagnosis. In any of the methods and treatments described herein, the animal or mammal can be in need thereof.

As used herein, the phrase “integer from 1 to 5” means 1, 2, 3, 4, or 5.

As used herein, the term “mammal” means a rodent (i.e., a mouse, a rat, or a guinea pig), a monkey, a cat, a dog, a cow, a horse, a pig, or a human. In some embodiments, the mammal is a human.

As used herein, the term “n-membered”, where n is an integer, typically describes the number of ring-forming atoms in a moiety, where the number of ring-forming atoms is n. For example, pyridine is an example of a 6-membered heteroaryl ring and thiophene is an example of a 5-membered heteroaryl ring.

As used herein, the phrase “optionally substituted” means that substitution is optional and therefore includes both unsubstituted and substituted atoms and moieties. A “substituted” atom or moiety indicates that any hydrogen on the designated atom or moiety can be replaced with a selection from the indicated substituent groups, provided that the normal valency of the designated atom or moiety is not exceeded, and that the substitution results in a stable compound. For example, if a methyl group is optionally substituted, then 3 hydrogen atoms on the carbon atom can be replaced with substituent groups.

As used herein, the phrase “pharmaceutically acceptable” means those compounds, materials, compositions, and/or dosage forms which are, within the scope of sound medical judgment, suitable for use in contact with tissues of humans and animals. In some embodiments, “pharmaceutically acceptable” means approved by a regulatory agency of the Federal or a state government or listed in the U.S. Pharmacopeia or other generally recognized pharmacopeia for use in animals, and more particularly in humans.

As used herein, the phrase “pharmaceutically acceptable salt(s),” includes, but is not limited to, salts of acidic or basic groups. Compounds that are basic in nature are capable of forming a wide variety of salts with various inorganic and organic acids. Acids that may be used to prepare pharmaceutically acceptable acid addition salts of such basic compounds are those that form non-toxic acid addition salts, i.e., salts containing pharmacologically acceptable anions including, but not limited to, sulfuric, thiosulfuric, citric, malic, maleic, acetic, oxalic, hydrochloride, hydrobromide, hydroiodide, nitrate, sulfate, bisulfate, bisulfite, phosphate, acid phosphate, isonicotinate, borate, acetate, lactate, salicylate, citrate, acid citrate, tartrate, oleate, tannate, pantothenate, bitartrate, ascorbate, succinate, malate, maleate, gentisinate, fumarate, gluconate, glucaronate, saccharate, formate, benzoate, glutamate, methanesulfonate, ethanesulfonate, benzenesulfonate, p-toluenesulfonate, bicarbonate, malonate, mesylate, esylate, napsydisylate, tosylate, besylate, orthophoshate, trifluoroacetate, and pamoate (i.e., 1,1′-methylene-bis-(2-hydroxy-3-naphthoate)) salts. Compounds that include an amino moiety may form pharmaceutically acceptable salts with various amino acids, in addition to the acids mentioned above. Compounds that are acidic in nature are capable of forming base salts with various pharmacologically acceptable cations. Examples of such salts include, but are not limited to, alkali metal or alkaline earth metal salts and, particularly, calcium, magnesium, ammonium, sodium, lithium, zinc, potassium, and iron salts. The present invention also includes quaternary ammonium salts of the compounds described herein, where the compounds have one or more tertiary amine moiety.

As used herein, the term “phenyl” means —C₆H₅. A phenyl group can be unsubstituted or substituted with one, two, or three suitable substituents.

As used herein, the terms “prevention” or “preventing” mean a reduction of the risk of acquiring a particular disease, condition, or disorder.

As used herein, the phrase “suitable substituent” or “substituent” means a group that does not nullify the synthetic or pharmaceutical utility of the compounds described herein or the intermediates useful for preparing them. Examples of suitable substituents include, but are not limited to: C₁₋₆alkyl, C₁₋₆alkenyl, C₁₋₆alkynyl, C₅-C₆aryl, C₁₋₆alkoxy, C₃-C₅heteroaryl, C₃-C₆cycloalkyl, C₅-C₆aryloxy, —CN, —OH, oxo, halo, haloalkyl, —NO₂, —CO₂H, —NH₂, —CHO, —NH(C₁-C₈alkyl), —N(C₁-C₈alkyl)₂, —NH(C₆aryl), —N(C₅-C₆aryl)₂, —CO(C₁-C₆alkyl), —CO((C₅-C₆)aryl), —CO₂((C₁-C₆)alkyl), and —CO₂((C₅-C₆)aryl). One of skill in art can readily choose a suitable substituent based on the stability and pharmacological and synthetic activity of the compounds described herein.

As used herein, the phrase “therapeutically effective amount” means the amount of active compound or pharmaceutical agent that elicits the biological or medicinal response that is being sought in a tissue, system, animal, individual or human by a researcher, veterinarian, medical doctor or other clinician. The therapeutic effect is dependent upon the disorder being treated or the biological effect desired. As such, the therapeutic effect can be a decrease in the severity of symptoms associated with the disorder and/or inhibition (partial or complete) of progression of the disorder, or improved treatment, healing, prevention or elimination of a disorder, or side-effects, or at least one adverse effect of a disorder is ameliorated or alleviated. The amount needed to elicit the therapeutic response can be determined based on the age, health, size and sex of the subject. Optimal amounts can also be determined based on monitoring of the subject's response to treatment.

As used herein, the terms “treat,” “treated,” or “treating” mean therapeutic treatment measures wherein the object is to slow down (lessen) an undesired physiological condition, disorder or disease, or obtain beneficial or desired clinical results. Beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment may include eliciting a clinically significant response without excessive levels of side effects. Treatment may also include prolonging survival as compared to expected survival if not receiving treatment.

The compounds of the disclosure are identified herein by their chemical structure and/or chemical name. Where a compound is referred to by both a chemical structure and a chemical name, and that chemical structure and chemical name conflict, the chemical structure is determinative of the compound's identity.

At various places in the present specification, substituents of compounds may be disclosed in groups or in ranges. It is specifically intended that the invention include each and every individual subcombination of the members of such groups and ranges. For example, the term “C₁₋₆alkyl” is specifically intended to individually disclose methyl, ethyl, propyl, C₄alkyl, C₅alkyl, and C₆alkyl, linear and/or branched.

For compounds in which a variable appears more than once, each variable can be a different moiety selected from the Markush group defining the variable. For example, where a structure is described having two R groups that are simultaneously present on the same compound, the two R groups can represent different moieties selected from the Markush groups defined for R. In another example, when an optionally multiple substituent is designated in the form, for example,

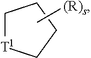

then it is understood that substituent R can occur “s” number of times on the ring, and R can be a different moiety at each occurrence. Further, in the above example, where the variable T¹ is defined to include hydrogens, such as when T¹ is CH₂, NH, etc., any H can be replaced with a substituent.

It is further appreciated that certain features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable sub-combination.

It is understood that the present disclosure encompasses the use, where applicable, of stereoisomers, diastereomers and optical stereoisomers of the compounds of the disclosure, as well as mixtures thereof. Additionally, it is understood that stereoisomers, diastereomers, and optical stereoisomers of the compounds of the disclosure, and mixtures thereof, are within the scope of the disclosure. By way of non-limiting example, the mixture may be a racemate or the mixture may comprise unequal proportions of one particular stereoisomer over the other. Additionally, the compounds can be provided as a substantially pure stereoisomers, diastereomers and optical stereoisomers (such as epimers).

The compounds described herein may be asymmetric (e.g., having one or more stereocenters). All stereoisomers, such as enantiomers and diastereomers, are intended to be included within the scope of the disclosure unless otherwise indicated. Compounds that contain asymmetrically substituted carbon atoms can be isolated in optically active or racemic forms. Methods of preparation of optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds are also included within the scope of the disclosure and can be isolated as a mixture of isomers or as separated isomeric forms. Where a compound capable of stereoisomerism or geometric isomerism is designated in its structure or name without reference to specific R/S or cis/trans configurations, it is intended that all such isomers are contemplated.

Resolution of racemic mixtures of compounds can be carried out by any of numerous methods known in the art, including, for example, fractional recrystallizaion using a chiral resolving acid which is an optically active, salt-forming organic acid. Suitable resolving agents for fractional recrystallization methods include, but are not limited to, optically active acids, such as the D and L forms of tartaric acid, diacetyltartaric acid, dibenzoyltartaric acid, mandelic acid, malic acid, lactic acid, and the various optically active camphorsulfonic acids such as β-camphorsulfonic acid. Other resolving agents suitable for fractional crystallization methods include, but are not limited to, stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like. Resolution of racemic mixtures can also be carried out by elution on a column packed with an optically active resolving agent (e.g., dinitrobenzoylphenylglycine). Suitable elution solvent compositions can be determined by one skilled in the art.

Compounds may also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Examples of prototropic tautomers include, but are not limited to, ketone-enol pairs, amide-imidic acid pairs, lactam-lactim pairs, amide-imidic acid pairs, enamine-imine pairs, and annular forms where a proton can occupy two or more positions of a heterocyclic system including, but not limited to, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds also include hydrates and solvates, as well as anhydrous and non-solvated forms.

Compounds can also include all isotopes of atoms occurring in the intermediates or final compounds. Isotopes include those atoms having the same atomic number but different mass numbers. For example, isotopes of hydrogen include tritium and deuterium.

In some embodiments, the compounds, or pharmaceutically acceptable salts thereof, are substantially isolated. Partial separation can include, for example, a composition enriched in the compound of the disclosure. Substantial separation can include compositions containing at least about 50%, at least about 60%, at least about 70%, at least about 80%, at least about 90%, at least about 95%, at least about 97%, or at least about 99% by weight of the compound of the disclosure, or pharmaceutically acceptable salt thereof. Methods for isolating compounds and their salts are routine in the art.

Although the disclosed compounds are suitable, other functional groups can be incorporated into the compound with an expectation of similar results. In particular, thioamides and thioesters are anticipated to have very similar properties. The distance between aromatic rings can impact the geometrical pattern of the compound and this distance can be altered by incorporating aliphatic chains of varying length, which can be optionally substituted or can comprise an amino acid, a dicarboxylic acid or a diamine. The distance between and the relative orientation of monomers within the compounds can also be altered by replacing the amide bond with a surrogate having additional atoms. Thus, replacing a carbonyl group with a dicarbonyl alters the distance between the monomers and the propensity of dicarbonyl unit to adopt an anti arrangement of the two carbonyl moiety and alter the periodicity of the compound. Pyromellitic anhydride represents still another alternative to simple amide linkages which can alter the conformation and physical properties of the compound. Modern methods of solid phase organic chemistry now allow the synthesis of homodisperse compounds with molecular weights approaching 5,000 Daltons. Other substitution patterns are equally effective.

The compounds described herein also include derivatives referred to as prodrugs, which can be prepared by modifying functional groups present in the compounds in such a way that the modifications are cleaved, either in routine manipulation or in vivo, to the parent compounds. Examples of prodrugs include compounds as described herein that contain one or more molecular moieties appended to a hydroxyl, amino, sulfhydryl, or carboxyl group of the compound, and that when administered to a patient, cleaves in vivo to form the free hydroxyl, amino, sulfhydryl, or carboxyl group, respectively. Examples of prodrugs include, but are not limited to, acetate, formate and benzoate derivatives of alcohol and amine functional groups in the compounds described herein.

Compounds containing an amine function can also form N-oxides. A reference herein to a compound that contains an amine function also includes the N-oxide. Where a compound contains several amine functions, one or more than one nitrogen atom can be oxidized to form an N-oxide. Examples of N-oxides include N-oxides of a tertiary amine or a nitrogen atom of a nitrogen-containing heterocycle. N-Oxides can be formed by treatment of the corresponding amine with an oxidizing agent such as hydrogen peroxide or a per-acid (e.g., a peroxycarboxylic acid).

The present disclosure provides methods of treating liver diseases, such as NASH, NAFLD, FALD, alcoholic liver disease, and/or liver fibrosis in a mammal in need thereof, comprising administering to the mammal any one or more of the lyn kinase activators described herein, or compositions comprising the same. In some embodiments, a mammal, such as a human, having NASH is treated. In some embodiments, a mammal, such as a human, having NAFLD is treated. In some embodiments, a mammal, such as a human, having liver fibrosis is treated. In some embodiments, a mammal, such as a human, having FALD is treated. In some embodiments, a mammal, such as a human, having alcoholic liver disease is treated.

In some embodiments, the lyn kinase activator is of the formula:

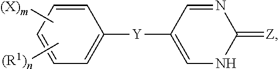

wherein: R¹ is an alkyl group; X is a halogen; Y is O, S, or NH; Z is O or S; and n is an integer from 0 to 5 and m is 0 or 1, wherein m+n is less than or equal to 5; or a pharmaceutically acceptable salt thereof. In some embodiments, the alkyl group is methyl and n is 1. In some embodiments, the halogen is chlorine and m is 1. In some embodiments, Y is O. In some embodiments, Z is O. In some embodiments, R is methyl, Y is O, Z is O, n is 1, and m is 0. In some embodiments, R is in the meta position. In some embodiments, X is chlorine, Y is O, Z is O, n is 0, and m is 1. In some embodiments, X is in the meta position.

In some embodiments, the lyn kinase activator is of the formula:

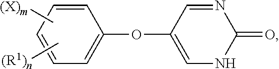

wherein: R¹ is an alkyl group; X is a halogen; and n is an integer from 0 to 5 and m is 0 or 1, wherein m+n is less than or equal to 5; or a pharmaceutically acceptable salt thereof. In some embodiments, the alkyl group is methyl and n is 1. In some embodiments, the halogen is chlorine and m is 1. In some embodiments, R¹ is methyl, n is 1, and m is 0. In some embodiments, R¹ is in the meta position. In some embodiments, X is chlorine, n is 0, and m is 1. In some embodiments, X is in the meta position.

In some embodiments, the lyn kinase activator is of the formula:

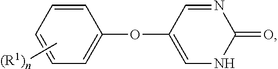

wherein R¹ is an alkyl group and n is an integer from 0 to 5; or a pharmaceutically acceptable salt thereof. In some embodiments, R¹ is methyl, n is 1. In some embodiments, R¹ is in the meta position.

In some embodiments, the lyn kinase activator is of the formula:

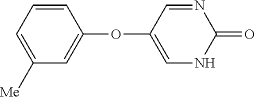 [SMILES: Cc1cccc(Oc2cnc(=O)[nH]c2)c1]

(Compound 102; MLR-1023; tolimidone), or a pharmaceutically acceptable salt thereof.

In some embodiments, the lyn kinase activator is of the formula:

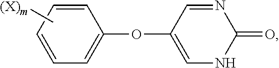

wherein X is a halogen and m is an integer from 0 to 1; or a pharmaceutically acceptable salt thereof. In some embodiments, X is chloro and m is 1. In some embodiments, X is in the meta position.

In some embodiments, the lyn kinase activator is of the formula:

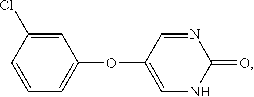 [SMILES: O=c1ncc(Oc2cccc(Cl)c2)c[nH]1]

or a pharmaceutically acceptable salt thereof.

In some embodiments, the lyn kinase activator is of the formula:

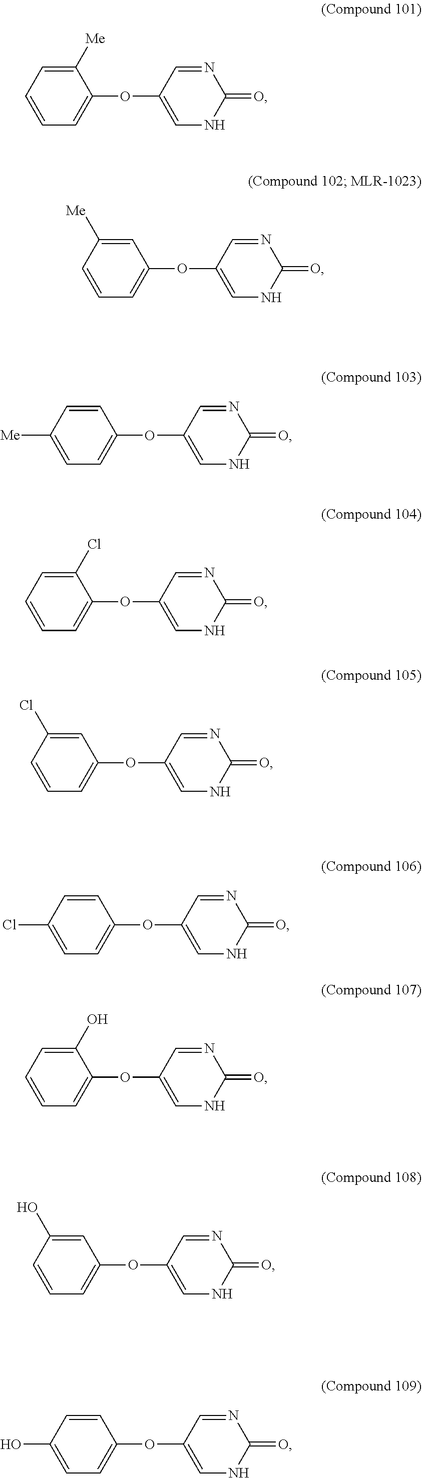

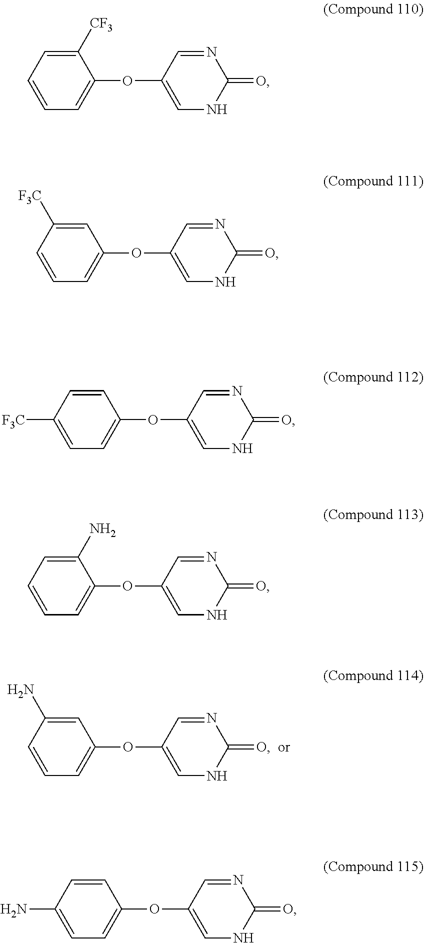 [SMILES: O=c1ncc(Oc2ccccc2C(F)(F)F)c[nH]1 ; O=c1ncc(Oc2cccc(C(F)(F)F)c2)c[nH]1 ; O=c1ncc(Oc2ccc(C(F)(F)F)cc2)c[nH]1 ; Nc1ccccc1Oc1cnc(=O)[nH]c1 ; Nc1cccc(Oc2cnc(=O)[nH]c2)c1 ; Nc1ccc(Oc2cnc(=O)[nH]c2)cc1]

or a pharmaceutically acceptable salt thereof.

In some embodiments, the lyn kinase activator is of the formula:

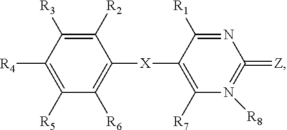

wherein: each of R₁, R₂, R₃, R₄, R₅, R₆, and R₇ is, independently, a hydrogen, alkoxy, alkyl, alkenyl, alkynyl, aryl, aryloxy, benzyl, cycloalkyl, halogen, heteroaryl, heterocycloalkyl, —CN, —OH, —NO₂, —CF₃, —CO₂H, —CO₂alkyl, or —NH₂; R₈ is an alkyl or hydrogen; X is O, S, NH, or N-alkyl; and Z is O or S; or a pharmaceutically acceptable salt thereof. In some embodiments, R₈ is alkyl. In some embodiments, R₈ is methyl. In some embodiments, R₈ is hydrogen. In some embodiments, X is oxygen. In some embodiments, Z is oxygen. In some embodiments, at least one of R₂-R₆ is alkyl. In some embodiments, at least one of R₂-R₆ is methyl. In some embodiments, at least one of R₂-R₆ is halogen. In some embodiments, at least one of R₂-R₆ is chloro. In some embodiments, at least one of R₂-R₆ is —CN, —OH, —NO₂, —CF₃, —CO₂H, —NH₂, or alkoxy. In some embodiments, R₂ is alkyl, each of R₁ and R₃-R₈ is hydrogen, and X and Z are O. In some embodiments, R₂ is methyl. In some embodiments, R₂ is a halogen, each of R₁ and R₃-R₈ is hydrogen, and X and Z are O. In some embodiments, R₂ is chloro. In some embodiments, R₃ is alkyl, each of R₁, R₂ and R₄-R₈ is hydrogen, and X and Z are O. In some embodiments, R₃ is methyl. In some embodiments, R₃ is a halogen, each of R₁, R₂, and R₄-R₈ is hydrogen, and X and Z are O. In some embodiments, R₃ is chloro. In some embodiments, R₄ is alkyl, each of R₁-R₃ and R₅-R₈ is hydrogen, and X and Z are O. In some embodiments, R₄ is methyl. In some embodiments, R₄ is a halogen, each of R₁-R₃ and R₅-R₈ is hydrogen, and X and Z are O. In some embodiments, R₄ is chloro. In some embodiments, R₅ is —CF₃, each of R₁-R₄ and R₆-R₈ is hydrogen, and X and Z are O. In some embodiments, R₅ is —NH₂, each of R₁-R₄ and R₆-R₈ is hydrogen, and X and Z are O. In some embodiments, R₆ is —CF₃, each of R₁-R₅ and R₇-R₈ is hydrogen, and X and Z are O. In some embodiments, R₆ is —NH₂, each of R₁-R₅ and R₇-R₈ is hydrogen, and X and Z are O.

In some embodiments, the lyn kinase activator is of the formula:

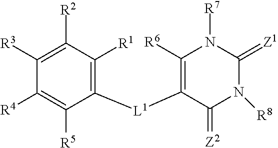

wherein:

R is H, halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, NR^{c1}S(O)₂NR^{c1}R^{d1}, S(O)R^{b1}, S(O)NR^{c1}R^{d1}, S(O)₂R^{b1}, NR^{c1}S(O)₂R^{b1}, or S(O)₂NR^{c1}R^{d1}, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4, or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2}.

R² is H, halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, NR^{c1}S(O)₂NR^{c1}R^{d1}, S(O)R^{b1}, S(O)NR^{c1}R^{d1}, S(O)₂R^{b1}, NR^{c1}S(O)₂R^{b1}, or S(O)₂NR^{c1}R^{d1}, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4, or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2};

R³ is H, halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, NR^{c1}S(O)₂NR^{c1}R^{d1}, S(O)R^{b1}, S(O)NR^{c1}R^{d1}, S(O)₂R^{b1}, NR^{c1}S(O)₂R^{b1}, or S(O)₂NR^{c1}R^{d1}, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4, or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2};

R⁴ is H, halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, NR^{c1}S(O)₂NR^{c1}R^{d1}, S(O)R^{b1}, S(O)NR^{c1}R^{d1}, S(O)₂R^{b1}, NR^{c1}S(O)₂R^{b1}, or S(O)₂NR^{c1}R^{d1}, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4, or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2};

R⁵ is H, halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, NR^{c1}S(O)₂NR^{c1}R^{d1}, S(O)R^{b1}, S(O)NR^{c1}R^{d1}, S(O)₂R^{b1}, NR^{c1}S(O)₂R^{b1}, or S(O)₂NR^{c1}R^{d1}, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4, or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2};
or two adjacent groups of R¹, R², R³, R⁴, and R⁵ can link to form a fused cycloalkyl or fused heterocycloalkyl group, each optionally substituted by 1, 2, or 3 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2};

R⁶ is H, halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, NR^{c1}S(O)₂NR^{c1}R^{d1}, S(O)R^{b1}, S(O)NR^{c1}R^{d1}, S(O)₂R^{b1}, NR^{c1}S(O)₂R^{b1}, or S(O)₂NR^{c1}R^{d1}, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4, or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2}.

R⁷ is H, C₁₋₆alkyl, C₁₋₆haloalkyl, C₁₋₆hydroxyalkyl, C(O)R^{b1}, C(O)NR^{c1}R^{d1}, C(O)OR^{a1}, S(O)R^{b1}, S(O)NR^{c1}R^{d1}, S(O)₂R^{b1}, or S(O)₂NR^{c1}R^{d1}.

R⁸ is H, C₁₋₆alkyl, C₁₋₆haloalkyl, C₁₋₆hydroxyalkyl, C(O)R^{b1}, C(O)NR^{c1}R^{d1}, C(O)OR^{a1}, S(O)R^{b1}, S(O)NR^{c1}R^{d1}, S(O)₂R^{b1}, or S(O)₂NR^{c1}R^{d1}.

R^{a1}, R^{b1}, R^{c1}, and R^{d1} are each, independently, selected from H, C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, and heterocycloalkylalkyl, wherein each of C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, and heterocycloalkylalkyl is optionally substituted with 1, 2, 3, 4, or 5 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy;

or R^{c1} and R^{d1} together with the N atom to which they are attached form a 4-, 5-, 6-, or 7-membered heterocycloalkyl group or heteroaryl group, each optionally substituted with 1, 2, or 3 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy;

R^{a2}, R^{b2}, R^{c2}, and R^{d2} are each, independently, selected from H, C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, and heterocycloalkylalkyl, wherein each of C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, or heterocycloalkylalkyl is optionally substituted with 1, 2, or 3 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy;

or R^{c2} and R^{d2} together with the N atom to which they are attached form a 4-, 5-, 6-, or 7-membered heterocycloalkyl group or heteroaryl group, each optionally substituted with 1, 2, or 3 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy;

Z¹ is O, S, or NR⁹;

R⁹ is H, OH, C₁₋₆alkoxy, C₁₋₆haloalkyl, C₁₋₆hydroxyalkyl, aryloxy, heteroaryloxy, CN, or NO₂;

Z² is O, S, or NR¹⁰;

R¹⁰ is H, OH, C₁₋₆alkoxy, C₁₋₆haloalkyl, C₁₋₆hydroxyalkyl, aryloxy, heteroaryloxy, CN, or NO₂;

L¹ is O, S, or NR¹¹; and

R¹¹ is H, C₁₋₆alkyl, C₁₋₆haloalkyl, C₁₋₆hydroxyalkyl, C(O)R^{b1}, C(O)NR^{c1}R^{d1}, C(O)OR^{a1}, S(O)R^{b1}, S(O)NR^{c1}R^{d1}, S(O)₂R^{b1}, or S(O)₂NR^{c1}R^{d1}; or a pharmaceutically acceptable salt thereof.

In some embodiments, the lyn kinase activator is of the formula:

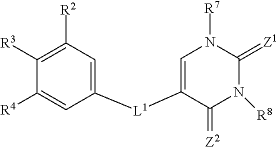

wherein:

R², R³ and R⁴ are each, independently, H, halo, C₁₋₆alkyl, C₁₋₆hydroxyalkyl, or C₁₋₆haloalkyl;

R⁷ is H, C₁₋₆alkyl, C(O)R^{b1}, C(O)NR^{c1}R^{d1}, or C(O)OR^{a1};

R⁸ is H, C₁₋₆alkyl, C(O)R^{b1}, C(O)NR^{c1}R^{d1}, or C(O)OR^{a1};

R^{a1}, R^{b1}, R^{c1}, and R^{d1} are each, independently, selected from H, C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, and heterocycloalkylalkyl, wherein each of C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, and heterocycloalkylalkyl is optionally substituted with 1, 2, 3, 4, or 5 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy;

or R^{c1} and R^{d1} together with the N atom to which they are attached form a 4-, 5-, 6-, or 7-membered heterocycloalkyl group or heteroaryl group, each optionally substituted with 1, 2, or 3 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy;

Z¹ is O or S;

Z² is O or S; and

L¹ is O or S; or a pharmaceutically acceptable salt thereof.

In some embodiments, the lyn kinase activator is of the formula:

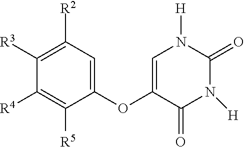

wherein: R², R³, R⁴, and R⁵ are each, independently, H, F, Cl, CH₃, SCH₃, OCH₃, C(CH₃)₃, CH(CH₃)₂, or C₂H₅; or a pharmaceutically acceptable salt thereof.

In some embodiments, the lyn kinase activator is of the formula:

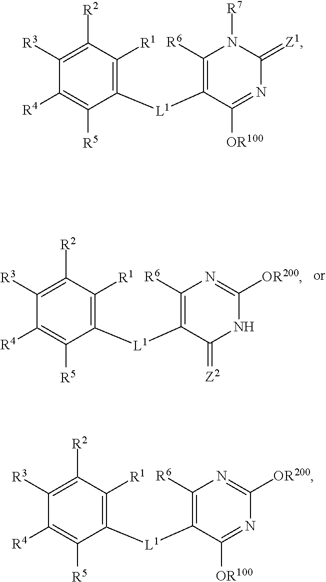

wherein:

R¹ is H, halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, NR^{c1}S(O)₂NR^{c1}R^{d1}, S(O)R^{b1}, S(O)NR^{c1}R^{d1}, S(O)₂R^{b1}, NR^{c1}S(O)₂R^{b1}, or S(O)₂NR^{c1}R^{d1}, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4, or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2};

R² is H, halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, NR^{c1}S(O)₂NR^{c1}R^{d1}, S(O)R^{b1}, S(O)NR^{c1}R^{d1}, S(O)₂R^{b1}, NR^{c1}S(O)₂R^{b1}, or S(O)₂NR^{c1}R^{d1}, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4, or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2}.

R³ is H, halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, NR^{c1}S(O)₂NR^{c1}R^{d1}, S(O)R^{b1}, S(O)NR^{c1}R^{d1}, S(O)₂R^{b1}, NR^{c1}S(O)₂R^{b1}, or S(O)₂NR^{c1}R^{d1}, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4, or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2};

R⁴ is H, halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, NR^{c1}S(O)₂NR^{c1}R^{d1}, S(O)R^{b1}, S(O)NR^{c1}R^{d1}, S(O)₂R^{b1}, NR^{c1}S(O)₂R^{b1}, or S(O)₂NR^{c1}R^{d1}, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4, or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2};

R⁵ is H, halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, NR^{c1}S(O)₂NR^{c1}R^{d1}, S(O)R^{b1}, S(O)NR^{c1}R^{d1}, S(O)₂R^{b1}, NR^{c1}S(O)₂R^{b1}, or S(O)₂NR^{c1}R^{d1}, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4, or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2};

or two adjacent groups of R¹, R², R³, R⁴, and R⁵ can link to form a fused cycloalkyl or fused heterocycloalkyl group, each optionally substituted by 1, 2, or 3 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2};

R⁶ is H, halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, NR^{c1}S(O)₂NR^{c1}R^{d1}, S(O)R^{b1}, S(O)NR^{c1}R^{d1}, S(O)₂R^{b1}, NR^{c1}S(O)₂R^{b1}, or S(O)₂NR^{c1}R^{d1}, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4, or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2}.

R⁷ is H, C₁₋₆alkyl, C₁₋₆haloalkyl, C(O)R^{b1}, C(O)NR^{c1}R^{d1}, C(O)OR^{a1}, S(O)R^{b1}, S(O)NR^{c1}R^{d1}, S(O)₂R^{b1}, or S(O)₂NR^{c1}R^{d1}.

R^{a1}, R^{b1}, R^{c1}, and R^{d1} are each, independently, selected from H, C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, and heterocycloalkylalkyl, wherein each of C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, and heterocycloalkylalkyl is optionally substituted with 1, 2, 3, 4, or 5 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy;

or R^{c1} and R^{d1} together with the N atom to which they are attached form a 4-, 5-, 6-, or 7-membered heterocycloalkyl group or heteroaryl group, each optionally substituted with 1, 2, or 3 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy;

R^{a2}, R^{b2}, R^{c2}, and R^{d2} are each, independently, selected from H, C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, and heterocycloalkylalkyl, wherein each of C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, or heterocycloalkylalkyl is optionally substituted with 1, 2, or 3 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy;

or R^{c2} and R^{d2} together with the N atom to which they are attached form a 4-, 5-, 6-, or 7-membered heterocycloalkyl group or heteroaryl group, each optionally substituted with 1, 2, or 3 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy;

Z¹ is O, S, or NR⁹;

R⁹ is H, OH, C₁₋₆alkoxy, C₁₋₆haloalkyl, C₁₋₆hydroxyalkyl, aryloxy, heteroaryloxy, CN, or NO₂;

Z² is O, S, or NR¹⁰;

R¹⁰ is H, OH, C₁₋₆alkoxy, C₁₋₆haloalkyl, C₁₋₆hydroxyalkyl, aryloxy, heteroaryloxy, CN, or NO₂;

L¹ is O, S, or NR¹¹;

R¹¹ is H, C₁₋₆alkyl, C₁₋₆haloalkyl, C₁₋₆hydroxyalkyl, C(O)R^{b1}, C(O)NR^{c1}R^{d1}, C(O)OR^{a1}, S(O)R^{b1}, S(O)NR^{c1}R^{d1}, S(O)₂R^{b1}, or S(O)₂NR^{c1}R^{d1}.

R¹⁰⁰ is a hydroxyl protecting group, C₁₋₆alkyl, C₂₋₆alkenyl, C₂₋₆alkynyl, C₁₋₆haloalkyl, C₁₋₆hydroxyalkyl, C₃₋₆cycloalkyl, heterocycloalkyl, aryl, heteroaryl, C(O)R^{b1}, C(O)NR^{c1}R^{d1}, C(O)OR^{a1}, S(O)R^{b1}, S(O)NR^{c1}R^{d1}, S(O)₂R^{b1}, S(O)₂NR^{c1}R^{d1}, S(O)₂OR^{e1}, P(O)OR^{f1}OR^{g1}, or Si(R^{h1})₃, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4 or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2}.

R²⁰⁰ is a hydroxyl protecting group, C₁₋₆alkyl, C₂₋₆alkenyl, C₂₋₆alkynyl, C₁₋₆haloalkyl, C₁₋₆hydroxyalkyl, C₃₋₆cycloalkyl, heterocycloalkyl, aryl, heteroaryl, C(O)R^{b1}, C(O)NR^{c1}R^{d1}, C(O)OR^{a1}, S(O)R^{b1}, S(O)NR^{c1}R^{d1}, S(O)₂R^{b1}, S(O)₂NR^{c1}R^{d1}, S(O)₂OR^{e1}, P(O)OR^{f1}OR^{g1}, or Si(R^{h1})₃, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4 or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2};

each R^{e} is, independently, H, C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, cycloalkylalkyl, arylalkyl, heterocycloalkylalkyl, or heteroarylalkyl;

each R^{f1} is, independently, H, C₁₋₆alkyl, C₁₋₆haloalkyl, C₁₋₆hydroxyalkyl, C₂₋₆alkenyl, (C₁₋₆alkoxy)-C₁₋₆alkyl, C₂₋₆alkynyl, aryl, cycloalkyl, heteroaryl, heterocycloalkyl, arylalkyl, cycloalkylalkyl, heteroarylalkyl, or heterocycloalkylalkyl;

each R^{g1} is, independently, H, C₁₋₆alkyl, C₁₋₆haloalkyl, C₁₋₆hydroxyalkyl, C₂₋₆alkenyl, C₂₋₆alkynyl, aryl, cycloalkyl, heteroaryl, or heterocycloalkyl; and

each R^{h1} is, independently, H, C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, cycloalkylalkyl, arylalkyl, heterocycloalkylalkyl, or heteroarylalkyl; or a pharmaceutically acceptable salt thereof.

In some embodiments, the lyn kinase activator is a compound of the formula:

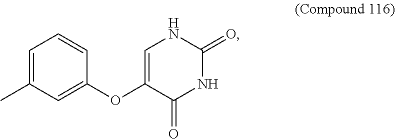 [SMILES: Cc1cccc(Oc2c[nH]c(=O)[nH]c2=O)c1]

which is also known as 5-(m-tolyloxy)pyrimidine-2,4(1H,3H)-dione.

It will be understood that the compounds are illustrative only and not intended to limit the scope of the claims to only those compounds.

The compounds described herein can be synthesized by standard organic chemistry techniques known to those of ordinary skill in the art, for example as described in U.S. Pat. Nos. 3,922,345 and 4,080,454. Preparation of the compounds described herein can involve the protection and deprotection of various chemical groups. The need for protection and deprotection, and the selection of appropriate protecting groups, can be readily determined by one skilled in the art. Suitable hydroxyl protecting groups include, but are not limited to, tert-butyldimethylsilyl (TBS), methoxymethyl ether (MOM), tetrahydropyranyl ether (THP), t-Butyl ether, allyl ether, benzyl ether, t-Butyldimethylsilyl ether (TBDMS), t-Butyldiphenylsilyl ether (TBDPS), acetic acid ester, and the like.

In some embodiments, the compositions described herein are pharmaceutical compositions and comprise a pharmaceutically acceptable carrier, vehicle, diluent, or excipient.

Vehicles include, but are not limited to a diluent, adjuvant, excipient, or carrier with which a compound is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used. When administered to a patient, the compounds and pharmaceutically acceptable vehicles are preferably sterile. Water is a suitable vehicle when the compound is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In some embodiments, the pharmaceutically acceptable vehicle is a capsule. Other examples of suitable pharmaceutical vehicles are described in Remington's Pharmaceutical Sciences, A. R. Gennaro (Editor) Mack Publishing Co.

The compounds can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The pharmaceutical compositions can also comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols. In some embodiments, the compounds described herein can be used with agents including, but not limited to, topical analgesics (e.g., lidocaine), barrier devices (e.g., GelClair), or rinses (e.g., Caphosol).

Suitable compositions include, but are not limited to, oral non-absorbed compositions. Suitable compositions also include, but are not limited to saline, water, cyclodextrin solutions, and buffered solutions of pH 3-9.

The compounds described herein, or pharmaceutically acceptable salts thereof, can be formulated with numerous excipients including, but not limited to, purified water, propylene glycol, PEG 400, glycerin, DMA, ethanol, benzyl alcohol, citric acid/sodium citrate (pH3), citric acid/sodium citrate (pH5), tris(hydroxymethyl)amino methane HCl (pH7.0), 0.9% saline, and 1.2% saline, and any combination thereof. In some embodiments, excipient is chosen from propylene glycol, purified water, and glycerin.

In some embodiments, the formulation can be lyophilized to a solid and reconstituted with, for example, water prior to use.

When administered to a human, the compounds can be sterile. Water is a suitable carrier when the compound is administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

In some embodiments, the compounds are formulated in accordance with routine procedures as a pharmaceutical composition adapted for administration to humans. Typically, compounds are solutions in sterile isotonic aqueous buffer. Where necessary, the compositions can also include a solubilizing agent. Compositions for intravenous administration may include a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the compound is to be administered by infusion, it can be dispensed, for example, with an infusion bottle containing sterile pharmaceutical grade water or saline. Where the compound is administered by injection, an ampoule of sterile water for injection or saline can be provided so that the ingredients may be mixed prior to administration.

The pharmaceutical compositions can be in unit dosage form. In such form, the composition can be divided into unit doses containing appropriate quantities of the active component. The unit dosage form can be a packaged preparation, the package containing discrete quantities of the preparations, for example, packeted tablets, capsules, and powders in vials or ampules. The unit dosage form can also be a capsule, cachet, or tablet itself, or it can be the appropriate number of any of these packaged forms.

In some embodiments, a composition can be in the form of a liquid wherein the active agent (i.e., one of the facially amphiphilic polymers or oligomers disclosed herein) is present in solution, in suspension, as an emulsion, or as a solution/suspension. In some embodiments, the liquid composition is in the form of a gel. In other embodiments, the liquid composition is aqueous. In other embodiments, the composition is in the form of an ointment.

Suitable preservatives include, but are not limited to, mercury-containing substances such as phenylmercuric salts (e.g., phenylmercuric acetate, borate and nitrate) and thimerosal; stabilized chlorine dioxide; quaternary ammonium compounds such as benzalkonium chloride, cetyltrimethylammonium bromide and cetylpyridinium chloride; imidazolidinyl urea; parabens such as methylparaben, ethylparaben, propylparaben and butylparaben, and salts thereof; phenoxyethanol; chlorophenoxyethanol; phenoxypropanol; chlorobutanol; chlorocresol; phenylethyl alcohol; disodium EDTA; and sorbic acid and salts thereof.

In some embodiments, one or more stabilizers can be included in the compositions to enhance chemical stability where required. Suitable stabilizers include, but are not limited to, chelating agents or complexing agents, such as, for example, the calcium complexing agent ethylene diamine tetraacetic acid (EDTA). For example, an appropriate amount of EDTA or a salt thereof, e.g., the disodium salt, can be included in the composition to complex excess calcium ions and prevent gel formation during storage. EDTA or a salt thereof can suitably be included in an amount of about 0.01% to about 0.5%. In those embodiments containing a preservative other than EDTA, the EDTA or a salt thereof, more particularly disodium EDTA, can be present in an amount of about 0.025% to about 0.1% by weight.

One or more antioxidants can also be included in the compositions. Suitable antioxidants include, but are not limited to, ascorbic acid, sodium metabisulfite, sodium bisulfite, acetylcysteine, polyquaternium-1, benzalkonium chloride, thimerosal, chlorobutanol, methyl paraben, propyl paraben, phenylethyl alcohol, edetate disodium, sorbic acid, or other agents know to those of skill in the art. Such preservatives are typically employed at a level of from about 0.001% to about 1.0% by weight.

In some embodiments, the compounds are solubilized at least in part by an acceptable solubilizing agent. Certain acceptable nonionic surfactants, for example polysorbate 80, can be useful as solubilizing agents, as can acceptable glycols, polyglycols, e.g., polyethylene glycol 400 (PEG-400), and glycol ethers. Suitable solubilizing agents for solution and solution/suspension compositions are cyclodextrins. Suitable cyclodextrins include α-cyclodextrin, β-cyclodextrin, γ-cyclodextrin, alkylcyclodextrins (such as, methyl-β-cyclodextrin, dimethyl-β-cyclodextrin, diethyl-β-cyclodextrin), hydroxyalkylcyclodextrins (such as, hydroxyethyl-β-cyclodextrin, hydroxypropyl-β-cyclodextrin), carboxy-alkylcyclodextrins (such as, carboxymethyl-β-cyclodextrin), sulfoalkylether cyclodextrins (such as, sulfobutylether-β-cyclodextrin), and the like. An acceptable cyclodextrin can optionally be present in a composition at a concentration from about 1 to about 200 mg/ml, from about 5 to about 100 mg/ml, or from about 10 to about 50 mg/ml.

In some embodiments, the composition contains a suspending agent. For example, in those embodiments in which the composition is an aqueous suspension or solution/suspension, the composition can contain one or more polymers as suspending agents. Useful polymers include, but are not limited to, water-soluble polymers such as cellulosic polymers, for example, hydroxypropyl methylcellulose, and water-insoluble polymers such as cross-linked carboxyl-containing polymers.

One or more acceptable pH adjusting agents and/or buffering agents can be included in the compositions, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In some embodiments, one or more acceptable surfactants, such as nonionic surfactants, or co-solvents can be included in the compositions to enhance solubility of the components of the compositions or to impart physical stability, or for other purposes. Suitable nonionic surfactants include, but are not limited to, polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40; polysorbate 20, 60 and 80; polyoxyethylene/polyoxypropylene surfactants (e.g., Pluronic® F-68, F84 and P-103); cyclodextrin; or other agents known to those of skill in the art. Typically, such co-solvents or surfactants are employed in the compositions at a level of from about 0.01% to about 2% by weight.

The compounds described herein can be formulated for parenteral administration by injection, such as by bolus injection or continuous infusion. The compounds can be administered by continuous infusion subcutaneously over a period of about 15 minutes to about 24 hours. Formulations for injection can be presented in unit dosage form, such as in ampoules or in multi-dose containers, with an added preservative. The compositions can take such forms as suspensions, solutions or emulsions in oily or aqueous vehicles, and can contain formulatory agents such as suspending, stabilizing and/or dispersing agents. In some embodiments, the injectable is in the form of short-acting, depot, or implant and pellet forms injected subcutaneously or intramuscularly. In some embodiments, the parenteral dosage form is the form of a solution, suspension, emulsion, or dry powder.

In some embodiments, the compounds are formulated in accordance with routine procedures as a pharmaceutical composition adapted for intravenous administration to human beings. Typically, compounds for intravenous administration are solutions in sterile isotonic aqueous buffer. Where necessary, the compositions may also include a solubilizing agent. Compositions for intravenous administration may optionally include a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the compound is to be administered by infusion, it can be dispensed, for example, with an infusion bottle containing sterile pharmaceutical grade water or saline. Where the compound is administered by injection, an ampoule of sterile water for injection or saline can be provided so that the ingredients may be mixed prior to administration.

The compounds described herein can also be formulated as a depot preparation. Such long acting formulations can be administered by implantation (for example subcutaneously or intramuscularly) or by intramuscular injection. Depot injections can be administered at about 1 to about 6 months or longer intervals. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

In some embodiments, the compositions can be administered orally. Compositions for oral delivery may be in the form of tablets, lozenges, aqueous or oily suspensions, granules, powders, emulsions, capsules, syrups, or elixirs, for example. Orally administered compositions may contain one or more additional agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions may be coated to delay disintegration and absorption in the gastrointestinal tract thereby providing a sustained action over an extended period of time. Selectively permeable membranes surrounding an osmotically active driving compound are also suitable for orally administered compounds. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles can be pharmaceutical grade.

For oral administration, the compounds described herein can be formulated by combining the compounds with pharmaceutically acceptable carriers. Such carriers enable the compounds to be formulated as tablets, pills, dragees, capsules, emulsions, liquids, gels, syrups, caches, pellets, powders, granules, slurries, lozenges, aqueous or oily suspensions, and the like, for oral ingestion by a patient to be treated. Pharmaceutical preparations for oral use can be obtained by, for example, adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Orally administered compositions can contain one or more optional agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions may be coated to delay disintegration and absorption in the gastrointestinal tract thereby providing a sustained action over an extended period of time. Selectively permeable membranes surrounding an osmotically active driving compound are also suitable for orally administered compounds. Oral compositions can include standard vehicles such as mannitol, lactose, starch, magnesium stearate, sodium saccharine, cellulose, magnesium carbonate, etc. Such vehicles are suitably of pharmaceutical grade.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added.

For buccal administration, the compositions can take the form of, such as, tablets or lozenges formulated in a conventional manner.

In some embodiments, the compounds can be delivered in a controlled release system. In some embodiments, a pump may be used. In some embodiments, polymeric materials can be used. In some embodiments, a controlled-release system can be placed in proximity of the target of the compounds described herein, such as the liver, thus requiring only a fraction of the systemic dose. In some embodiments, the compounds described herein can be delivered in a vesicle, in particular a liposome.

For administration by inhalation, the compounds described herein can be delivered in the form of an aerosol spray presentation from pressurized packs or a nebulizer, with the use of a suitable propellant, such as dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, such as gelatin for use in an inhaler or insufflator can be formulated containing a powder mix of the compound and a suitable powder base such as lactose or starch.

In transdermal administration, the compounds can be applied to a plaster, or can be applied by transdermal, therapeutic systems that are consequently supplied to the organism. In some embodiments, the compounds are present in creams, solutions, powders, fluid emulsions, fluid suspensions, semi-solids, ointments, pastes, gels, jellies, and foams, or in patches containing any of the same.

The amount of a lyn kinase activator that will be effective in the treatment of a particular disorder or condition disclosed herein will depend on the nature of the disorder or condition, and can be determined by standard clinical techniques. In addition, in vitro or in vivo assays may optionally be employed to help identify optimal dosage ranges. The precise dose to be employed in the compositions will also depend on the route of administration, and the seriousness of the disease or disorder, and should be decided according to the judgment of the practitioner and each patient's circumstances. However, suitable dosage ranges for oral administration are generally from about 0.001 mg to about 200 mg of a compound per kg body weight. In some embodiments, the oral dose is from about 0.01 mg to about 70 mg per kg body weight, from about 0.1 mg to about 50 mg per kg body weight, from about 0.5 mg to about 20 mg per kg body weight, from about 1 mg to about 10 mg per kg body weight, or about 5 mg of a compound per kg body weight. The dosage amounts described herein refer to total amounts administered; that is, if more than one compound is administered, the dosages correspond to the total amount of the compounds administered. Oral compositions can contain 10% to 95% active ingredient by weight. Suitable dosage ranges for oral administration are generally from about 50 μg to about 1,000 mg, from about 100 μg to about 500 mg, from about 250 μg to about 100 mg, from about 500 μg to about 50 mg, from about 1 mg to about 40 mg, from about 5 mg to about 25 mg, or from about 10 mg to about 20 mg.

Suitable dosage ranges for intravenous (i.v.) administration are from about 0.01 mg to about 100 mg per kg body weight, from about 0.1 mg to about 35 mg per kg body weight, and from about 1 mg to about 10 mg per kg body weight. Suitable dosage ranges for i.v. administration are generally from about 50 μg to about 1,000 mg, from about 100 μg to about 500 mg, from about 250 μg to about 100 mg, from about 500 μg to about 50 mg, from about 1 mg to about 40 mg, from about 5 mg to about 25 mg, or from about 10 mg to about 20 mg. Suitable dosage ranges for intranasal administration are generally from about 0.01 μg/kg body weight to about 1 mg/kg body weight. Recommended dosages for intradermal, intramuscular, intraperitoneal, subcutaneous, epidural, sublingual, intracerebral, intravaginal, transdermal administration or administration by inhalation are in the range of from about 0.001 mg to about 200 mg per kg of body weight. Suitable doses of the compounds for topical administration are in the range of about 0.001 mg to about 1 mg, depending on the area to which the compound is administered. Effective doses may be extrapolated from dose-response curves derived from in vitro or animal model test systems.

The present disclosure also provides pharmaceutical packs or kits comprising one or more containers filled with one or more compositions. In some embodiments, the container(s) can further contain a notice in the form prescribed by a governmental agency regulating the manufacture, use or sale of pharmaceuticals or biological products, which notice reflects approval by the agency of manufacture, use or sale for human administration. In some embodiments, the kit contains more than one lyn kinase activator.

In some embodiments, the compositions can be used in combination therapy with at least one other therapeutic agent. The compound and the additional therapeutic agent can act additively or synergistically. In some embodiments, a composition described herein is administered concurrently with the administration of another therapeutic agent, which can be part of the same composition as the compound or a different composition. In some embodiments, a composition described herein is administered prior or subsequent to administration of another therapeutic agent. As many of the disorders for which the compositions are useful in treating are chronic disorders, in some embodiments, the combination therapy involves alternating between administering a composition described herein and a composition comprising another therapeutic agent, e.g., to minimize the toxicity associated with a particular drug. The duration of administration of each drug or therapeutic agent can be, e.g., one month, three months, six months, or a year. In some embodiments, when a composition described herein is administered concurrently with another therapeutic agent that potentially produces adverse side effects including but not limited to toxicity, the therapeutic agent can advantageously be administered at a dose that falls below the threshold at which the adverse side is elicited.

The present compositions can also comprise, or be administered together or separately, with an additional therapeutic agent used to treat liver diseases. Examples of additional therapeutic agents suitable for use in treatment of liver diseases, such as NASH, that can be combined with one or more of the compounds described herein include, but are not limited to, OCALIVA® (obeticholic acid), Selonsertib, Elafibranor, Cenicriviroc, GR_MD_02, MGL_3196, IMM124E, ARAMCHOL™ (arachidyl amido cholanoic acid), GS0976, Emricasan, Volixibat, NGM282, GS9674, Tropifexor, MN_001, LMB763, BI_1467335, MSDC_0602, PF_05221304, DF102, Saroglitazar, BMS986036, Lanifibranor, Semaglutide, Nitazoxanide, GRI_0621, EYP001, VK2809, Nalmefene, LIK066, MT_3995, Elobixibat, Namodenoson, Foralumab, SAR425899, Sotagliflozin, EDP_305, Isosabutate, Gemcabene, TERN_101, KBP_042, PF_06865571, DUR928, PF_06835919, NGM313, BMS986171, Namacizumab, CER_209, ND_L02_s0201, RTU_1096, DRX_065, IONIS_DGAT2Rx, INT_767, NC_001, Seladepar, PXL770, TERN 201, NV556, AZD2693, SP_1373, VK0214, Hepastem, TGFTX4, RLBN1127, GKT 137831, RYI_018, CB4209-CB4211, and JH_0920.

The present compositions can also comprise, or be administered together or separately, with a statin. Statins include, but are not limited to, atorvastatin, pravastatin, fluvastatin, lovastatin, simvastatin, and cerivastatin.

The present compositions can also comprise, or be administered together or separately, with a PPAR agonist, for example a thiazolidinedione or a fibrate. Thiazolidinediones include, but are not limited to, 5-((4-(2-(methyl-2-pyridinylamino)ethoxy)phenyl)methyl)-2,4-thiazolidinedione, troglitazone, pioglitazone, ciglitazone, WAY-120,744, englitazone, AD 5075, darglitazone, and rosiglitazone. Fibrates include, but are not limited to, gemfibrozil, fenofibrate, clofibrate, or ciprofibrate. As mentioned previously, a therapeutically effective amount of a fibrate or thiazolidinedione often has toxic side effects. Accordingly, in some embodiments, when a composition described herein is administered in combination with a PPAR agonist, the dosage of the PPAR agonist is below that which is accompanied by toxic side effects.

The present compositions can also comprise, or be administered together or separately, with a bile-acid-binding resin. Bile-acid-binding resins include, but are not limited to, cholestyramine and colestipol hydrochloride.

The present compositions can also comprise, or be administered together or separately, with niacin or nicotinic acid.

The present compositions can also comprise, or be administered together or separately, with a RXR agonist. RXR agonists include, but are not limited to, LG 100268, LGD 1069, 9-cis retinoic acid, 2-(1-(3,5,5,8,8-pentamethyl-5,6,7,8-tetrahydro-2-naphthyl)-cyclopropyl)-pyridine-5-carboxylic acid, or 4-((3,5,5,8,8-pentamethyl-5,6,7,8-tetrahydro-2-naphthyl)2-carbonyl)-benzoic acid.

The present compositions can also comprise, or be administered together or separately, with a hormone. Hormones include, but are not limited to, thyroid hormone, estrogen and insulin. Suitable insulins include, but are not limited to, injectable insulin, transdermal insulin, inhaled insulin, or any combination thereof. As an alternative to insulin, an insulin derivative, secretagogue, sensitizer or mimetic may be used. Insulin secretagogues include, but are not limited to, forskolin, dibutryl cAMP or isobutylmethylxanthine (IBMX).

The present compositions can also comprise, or be administered together or separately, with a sulfonylurea-based drug. Sulfonylurea-based drugs include, but are not limited to, glisoxepid, glyburide, acetohexamide, chlorpropamide, glibornuride, tolbutamide, tolazamide, glipizide, gliclazide, gliquidone, glyhexamide, phenbutamide, and tolcyclamide.

The present compositions can also comprise, or be administered together or separately, with a biguanide. Biguanides include, but are not limited to, metformin, phenformin and buformin.

The present compositions can also comprise, or be administered together or separately, with an α-glucosidase inhibitor. α-glucosidase inhibitors include, but are not limited to, acarbose and miglitol.

The present compositions can also comprise, or be administered together or separately, with a cardiovascular drug. Cardiovascular drugs include, but are not limited to, peripheral anti-adrenergic drugs, centrally acting antihypertensive drugs (e.g., methyldopa, methyldopa HCl), antihypertensive direct vasodilators (e.g., diazoxide, hydralazine HCl), drugs affecting renin-angiotensin system, peripheral vasodilators, phentolamine, antianginal drugs, cardiac glycosides, inodilators (e.g., amrinone, milrinone, enoximone, fenoximone, imazodan, sulmazole), antidysrhythmic drugs, calcium entry blockers, ranitine, bosentan, and rezulin.

The present compositions can be administered together, or separately, with treatment with irradiation. For irradiation treatment, the irradiation can be gamma rays or X-rays.

The present compositions can also comprise, or be administered together or separately, with one or more chemotherapeutic agents. Useful chemotherapeutic agents include, but are not limited to, methotrexate, taxol, mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, and docetaxel. In some embodiments, a composition described herein further comprises one or more chemotherapeutic agents and/or is administered concurrently with radiation therapy. In some embodiments, chemotherapy or radiation therapy is administered prior or subsequent to administration of a present composition, at least an hour, five hours, 12 hours, a day, a week, a month, or several months (e.g., up to three months), subsequent to administration of a composition described herein.

The present compositions can also comprise, or be administered together or separately, with an FXR agonist such as, for example, OCALIVA® (obeticholic acid).

The present compositions can also comprise, or be administered together or separately, with a PPARα agonist such as, for example, fenofibrate.

The present compositions can also comprise, or be administered together or separately, with a GLP-1 agonists such as, for example, VICTOZA® or SAXENDA® (liraglutide), BYETTA® or BYDUREON® (exenatide), LYXUMIA® (lixisenatide), TANZEUM® (albiglutide), TRULICITY® (dulaglutide), and OZEMPIC® (semaglutide).

The present compositions can also comprise, or be administered together or separately, with a PPARα/δ dual agonist such as, for example, Elafibranor.

The present compositions can also comprise, or be administered together or separately, with an ACC inhibitor such as, for example, GS-0976.

The present compositions can also comprise, or be administered together or separately, with a CCR2/5 blocker such as, for example, Ceniciviroc (CVC).

The present compositions can also comprise, or be administered together or separately, with any one or more of the following: hexadecanoic acid, linoleic acid, phloretin, Vitamin D3, docosanoic acid, quercetin, D-erythro-sphingosine, ricinoleic acid, dodecanoic acid, gossypol, ellagic acid, damnacanthal, heptadecanoic acid, gamma-linolenic acid eicosanoic acid, arachidonic acid, pentacosanoic acid, hexacosanoic acid, dequalinium chloride, tetradecanoic acid, hispidin, tetracosanoic acid, tridecanoic acid, DL-3,4-dihydroxymandelic acid, pentadecanoic acid, ETYA, MNS, palmitoyl-DL-carnitine, adrenic acid, thiazolidinedione, heneicosanoic acid, tricosanoic acid, chelerythrine chloride, aminoindole, docosahexaenoic acid, 5-amino-2-methylindole, cobalt chloride (CoCl₂), piceatannol, eicosapentaenoic acid, sodium nitride (Na₃N), radicicol, safingol, myricitrin, 13-HODE, calcifediol, mead acid, 5-iodotubercidin, sphingosine-1-phosphate, docosadienoic acid, heptadecenoic acid, geldanamycin, calcitriol, eicosadienoic acid, melittin, 4-hydroxy-tamoxifen, herbimycin A, hydroxyeicosatetraenoic acid, ET-18-OCH₃, 15-HETE, 5-HETE, eicosatrienoic acid, bryostatin 1, ilmofosine, H-9, H-8, K-252c, HA-1004, K-252a, K-252b, HA-1077, 9-HODE, MDL-27032, UCN-01, bisindolylmaleimide V, calphostin C, 7-oxostaurosporine, bisindolylmaleimide VIII, lavendustin A, lavendustin C, KRIBB3, bisindolylmaleimide X, bisindolylmaleimide I, NGIC-I, Go 6976, bisindolylmaleimide III, bisindolylmaleimide II, bisindolylmaleimide VI, bisindolylmaleimide VII, dihydrochloride, Pp60 c-src, Ro-32-0432, Go 7874, fingolimod, enzastaurin, PP1, PP2, HA-100 dihydrochloride, PD 166285, PP1, 1-NM-PP1, CGP77675, PD 180970, dasatinib, PD173952, SU 6656, A-419259, saracatinib, bosutinib, sotrastaurin, KX1-004, CID 755673, ZM 306416, AZM 475271, WH-4-023, TC-S 7003, dasatinib monohydrate, TG 100572, A-770041, KX2-391, NVP-BHG712, ER 27319 maleate, TCS 21311, KB SRC 4, and PKC 20-28.

The present compositions can be administered orally. The compositions can also be administered by any other convenient route, for example, by infusion or bolus injection, by absorption through epithelial or mucocutaneous linings (e.g., oral mucosa, rectal and intestinal mucosa, etc.) and can be administered together with another biologically active agent. Administration can be systemic or local. Various delivery systems are known, e.g., encapsulation in liposomes, microparticles, microcapsules, capsules, etc., and can be used to administer the compositions. In some embodiments, more than one composition is administered to a patient. Methods of administration include, but are not limited to intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. The desired mode of administration is left to the discretion of the practitioner, and will depend in-part upon the site of the medical condition.

In some embodiments, it may be desirable to administer one or more compositions locally to the area in need of treatment. This may be achieved, for example, and not by way of limitation, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In some embodiments, administration can be by direct injection at the site (or former site) of an atherosclerotic plaque tissue.

Pulmonary administration can also be employed, e.g., by use of an inhaler or nebulizer, and formulation with an aerosolizing agent, or via perfusion in a fluorocarbon or synthetic pulmonary surfactant. In some embodiments, the compositions can be formulated as a suppository, with traditional binders and vehicles such as triglycerides.

The present disclosure also provides compositions described herein for use in treating liver diseases, such as NASH, NAFLD, FALD, alcoholic liver disease, and/or liver fibrosis in a mammal in need thereof.

The present disclosure also provides any one or more of the lyn kinase activators described herein for use in treating liver diseases, such as NASH, NAFLD, FALD, alcoholic liver disease, and/or liver fibrosis in a mammal in need thereof.

The present disclosure also provides compositions described herein for use in preparation of a medicament for treating liver diseases, such as NASH, NAFLD, FALD, alcoholic liver disease, and/or liver fibrosis in a mammal in need thereof.

The present disclosure also provides any one or more of the lyn kinase activators described herein for use in preparation of a medicament for treating liver diseases, such as NASH, NAFLD, FALD, alcoholic liver disease, and/or liver fibrosis in a mammal in need thereof.

In order that the subject matter disclosed herein may be more efficiently understood, examples are provided below. It should be understood that these examples are for illustrative purposes only and are not to be construed as limiting the claimed subject matter in any manner. Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

### EXAMPLES

### Example 1: Animal Studies

The animal model was performed essentially as described in Cong et al., Life Sciences, 2008, 82, 983-990. Briefly, male C57BL/6 mice at 6 weeks of age were fed, ad libitum, a modified high fat diet (mHFD) provided by Research Diets, and described in Cong et al. One group of control animals (n=11) remained on standard diet. All animals remained on their respective diets for 23 weeks (29 weeks of age). At that time, animals continued to remain on their respective diets and treatment regimens began. The group of animals on standard diet and one group of animals on mHFD (n=9) received vehicle administered by intraperitoneal injection, once daily. Two additional groups of animals on mHFD (n=8) received Compound 102 administration by intraperitoneal injection, once daily at either 30 mg/kg or 100 mg/kg. Compound 102 was formulated in, and the vehicle used was, 20% hydroxypropyl beta cyclodextrin (HPBCD).

Treatment regimen continued for an additional 8 weeks while the animals remained on their respective diets. At the end of 8 weeks of treatment, animals were fasted for 6 hours. After fasting, serum samples were collected. Animals were then sacrificed by cervical dislocation. Livers were harvested and processed for lipid extraction using Lipid Extraction Kit (Chloroform Free; BioVision Inc, Bilpitas, Calif.; Cat No K216-50).

Serum triglycerides and liver triglyceride were measured from the respective samples using Triglyceride assay kit (Wako Diagnostics, Richmond, Va.; Cat No 461-8992). Data were averaged and are expressed as the average SEM. Data were analyzed by two-way repeated measures ANOVA followed by a Dunnett's post hoc test. P values of less than 0.05 were considered to be statistically significant from control.

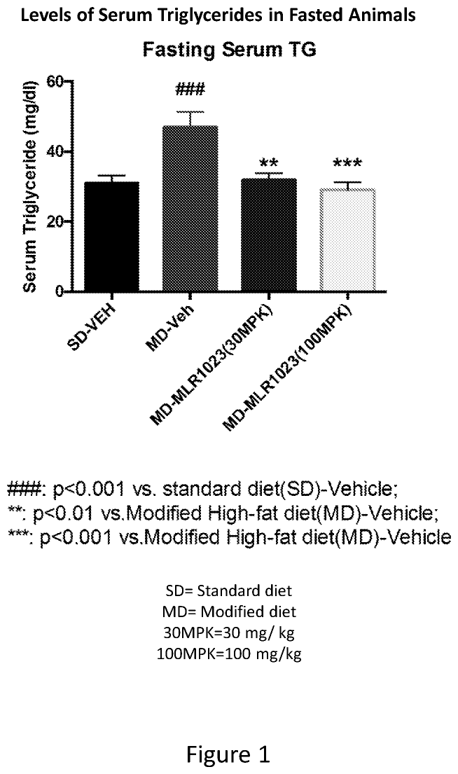

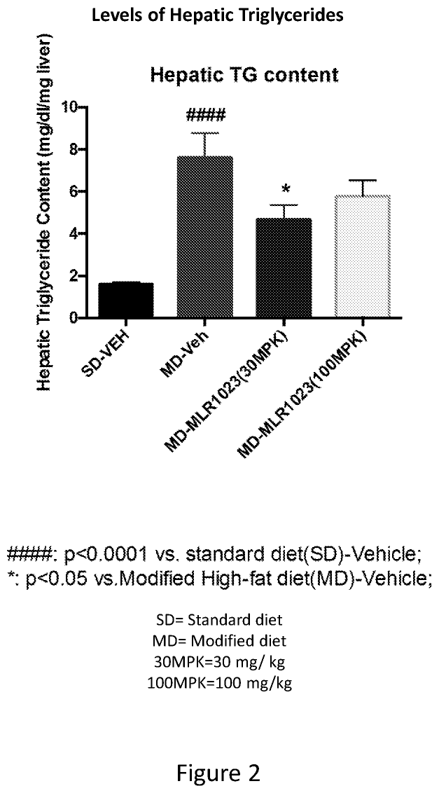

FIG. 1 shows that Compound 102 significantly reduced circulating serum triglyceride levels in animals on a high fat diet, and otherwise prone to exhibiting elevated triglycerides compared to animals on a normal diet. FIG. 2 shows that Compound 102 significantly reduced the accumulation of triglycerides in the liver on animals on a high fat diet and otherwise prone to accumulating elevated triglycerides compared to animals on a normal diet. These results indicate that Compound 102 may have therapeutic benefits towards diseases characterized by the accumulation of lipids in the liver including, but not limited to, non-alcoholic steatohepatisis (NASH), non-alcoholic fatty liver disease (NAFLD), fatty acid liver disease (FALD), alcoholic liver disease, and/or liver fibrosis.

### Example 2: Extended Animal Studies

### Materials and Methods

### Reagents:

The modified high fat diet (mHFD) was obtained from Research Diets, Inc. (New Brunswick, N.J.) and was formulated according to Cong et al., supra. The mHFD contained 60% fat, 14% protein, and 26% carbohydrate, with total energy content of 21.0 kJ/g. The mHFD contained a low concentration of choline bicitrate a (0.6 g/kg) and DL-methionine (1.5 g/kg). Fatty acid composition of the fats (mainly from lard) in mHFD was 36% saturated fatty acids, 45% monounsaturated fatty acids, and 19% polyunsaturated fatty acids (PUFA).

### Animals:

Male C57BL/6NCrl mice were obtained from Charles River Laboratories (Wilmington, Mass.). Mice were maintained in microisolator cages, in groups of no more than 4 animals per cage, and on a 12-hour light cycle. Food and water were provided ad libitum. On the last day of the study, all mice were sacrificed by cervical dislocation after a 4-hour food deprivation. Livers were rapidly dissected and part of each liver was cut and fixed in formaldehyde saline (4%) solution for histological analysis. The rest of the liver was snap frozen in liquid nitrogen, and stored at −70° C. until use.

### Experimental Protocol:

Mice were acquired at 3-4 weeks of age (body weight of about 11-12 g). Mice were fed a standard chow (Control group) or mHFD (Model group) for 22 weeks. During the 22-week diet induction period, weekly body weight and food intake were recorded to ensure that animal had a good tolerance or preference for the mHFD. At week-22, based on body weight consideration, the mHFD-fed mice were screened and distributed in a balanced fashion into 3 groups for the compound treatment study (Table 1).

TABLE 1
Study Design-Drug Treatment Phase

| | | | | DOSE |
|---|---|---|---|---|
| GROUP | | GROUP | DAYS OF | AND |
| # | TREATMENT | SIZE | DOSING: | ROUTE |
| 1 | Standard diet(SD)- | 10 | 56 | IP |
| | Vehicle | | | |
| 2 | mHFD(MD)- | 12 | 56 | IP |
| | Vehicle | | | |
| 3 | mHFD(MD)-MLR- | 12 | 56 | IP |
| | 1023 (30 mpk) | | | |
| 4 | mHFD(MD)-MLR- | 12 | 56 | IP |
| | 1023 (100 mpk) | | | |

During the 8-week drug treatment phase, all animals (except for standard-chow controls) remain on the mHFD diet. During the treatment period, the summary of measurement schedule is as below:
1) Body weight (BW) was taken once a week.
2) Terminal blood and whole Liver were collected.
3) Hepatic histology (NAS score) was assessed.

### Histology Analysis and NAFLD Activity Score (NAS):

Livers were removed at necropsy and fixed in 10% buffered formalin. Sections were processed to paraffin by routine histological methods and 4 μm sections were stained with hematoxylin and eosin. Sections were evaluated under light microscopy by a board-certified veterinary pathologist for identification of proliferative lesions as well as severity scoring of lesions associated with steatohepatitis. The pathologist was blinded to the treatment status. Steatohepatitis parameters were scored and classified according to a standardized histological scoring system for NASH described by Kleiner et al., Hepatology, 2005, 41, 1313-1321. In brief, individual livers were evaluated for steatosis, lobular inflammation, and ballooning degeneration of hepatocytes, and given a score of (03) for steatosis and inflammation, and (02) for ballooning degeneration, using previously defined criteria (see below). An NAFLD activity score (NAS) was obtained by summing the individual parameter scores.

### Scoring Criteria:

Steatosis Grade: 0: <5%; 1: 5%-33%; 2: >33%-66%; and 3: >66%.

Steatosis Location: 0: Predominantly zone 3 (centrilobular); 1: Predominantly zone 1 (periportal); 2: Azonal (defined as when the pattern could not be fit into one of the other categories or marked architectural change); and 3: Panacinar (defined as involving all zones of the liver equally).

Inflammation: 0: No foci; 1: <2 foci per 200× field; 2: 2-4 foci/200× field; and 3: >4 foci/200× field.

Ballooning Degeneration: 0: None; 1: Few balloon cells; and 2: Many cells/prominent ballooning.

### Analysis:

Data are expressed as the average±SEM at each week and analyzed by Unpaired T-tests as applicable using Prism® software (GraphPad Software, Inc., Las Jolla, Calif.). A p-value of less than 0.05 indicates a significant difference between treatment groups.

### Results

### Body Weight:

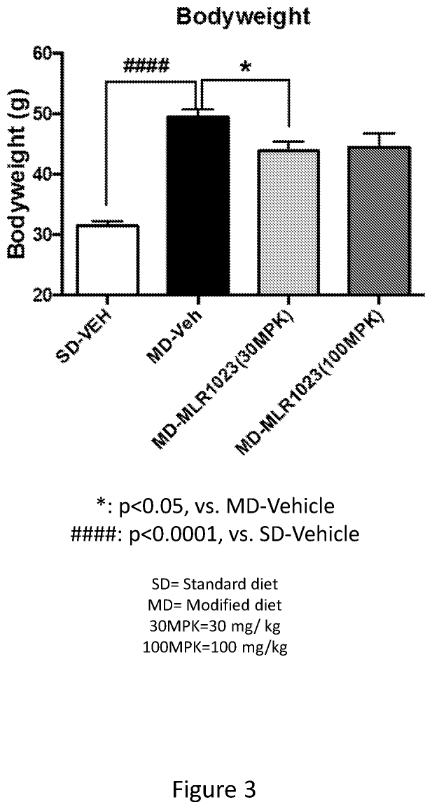

Body weights of all mice were measured at the end of study. Compared to Standard Diet-fed mice, MD-fed Vehicle mice had significantly higher body weights. Compared to MD-vehicle mice, 30 milligrams per kilogram (mpk) MLR-1023 significantly reduced the body weight after 8-week treatment. A trend in reduction (p=0.056) of body weights was observed in MLR-1023 (100 mpk) group, compared to MD-vehicle group (see, FIG. 3). All MD-fed mice/groups shared similar body weights before the initiation of drug treatment. Data are mean±SEM and analyzed by Unpaired T-tests as applicable.

### Liver Weight:

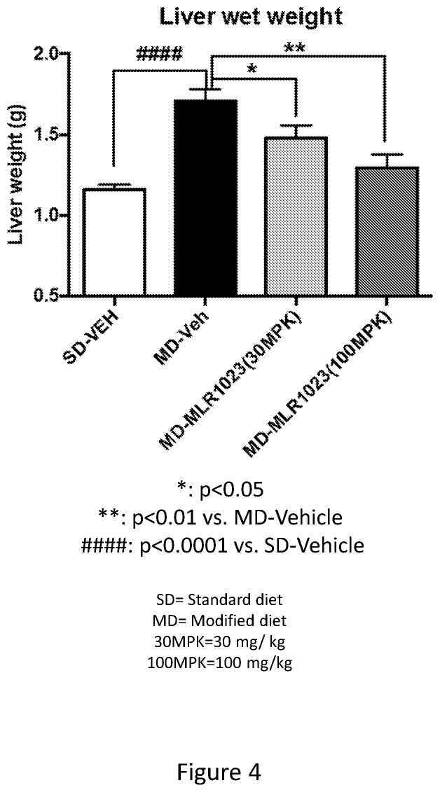

Liver wet weights of all mice were measured at the end of the study. Compared to SD-Vehicle mice, MD-Vehicle mice had significantly higher liver weights. Compared to MD-vehicle mice, 30 mpk MLR-1023 and 100 mpk MLR-1023 significantly reduced the liver weight after 8-week treatment (see, FIG. 4). Data are mean±SEM and analyzed by Unpaired T-tests as applicable. The reduction in liver weight demonstrated by MLR-1023 in this NASH model is an indicator for the reduction of liver fat and ultimately steatosis and NASH.

### Serum Lipid:

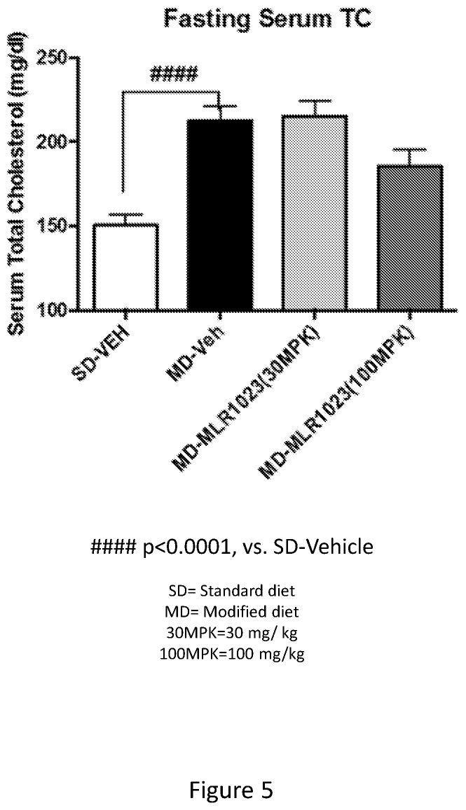

Fasting serum total cholesterol (TC) of all mice were measured at the end of study. No changes of serum TC levels were observed in MLR-1023-treated mice (see, FIG. 5). Data are mean±SEM and analyzed by Unpaired T-tests as applicable. The reduction of serum triglycerides (see, FIG. 1) represents therapeutic activity of MLR-1023 that is independent of its action as an insulin sensitizer, show MLR-1023's therapeutic potential on a frequently co-morbid condition associated with NASH (dyslipidemia), and provides an indirect indication that MLR-1023 may help reduce liver fat and steatosis.

### Hepatic TG and NAS Score

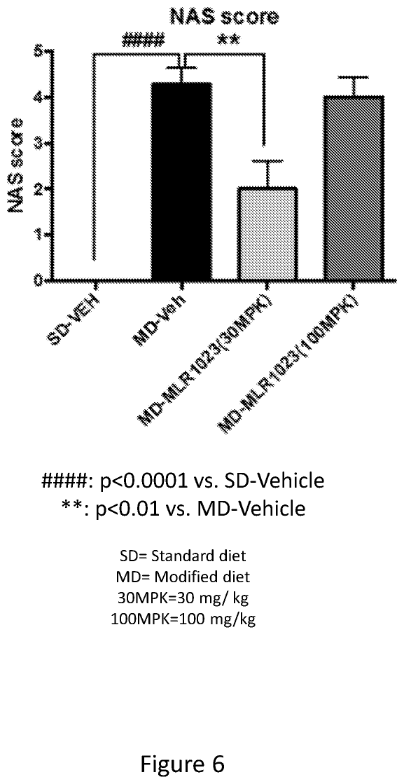

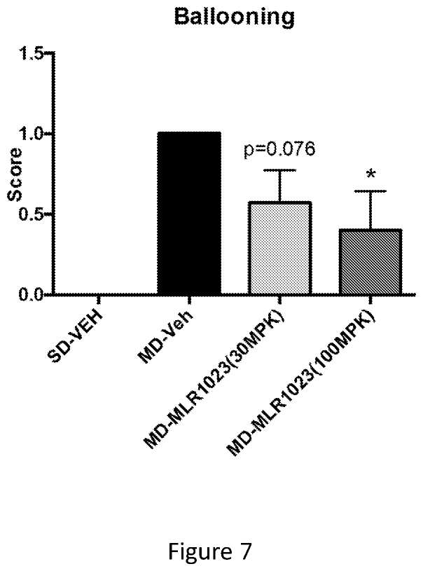

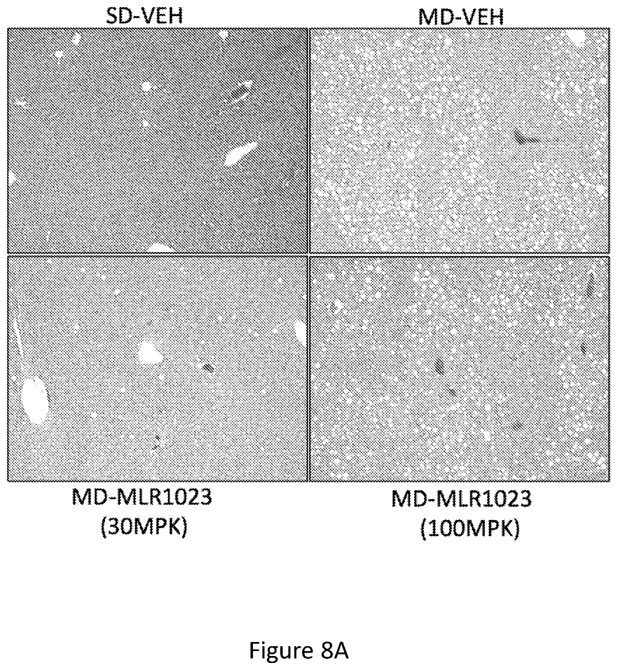

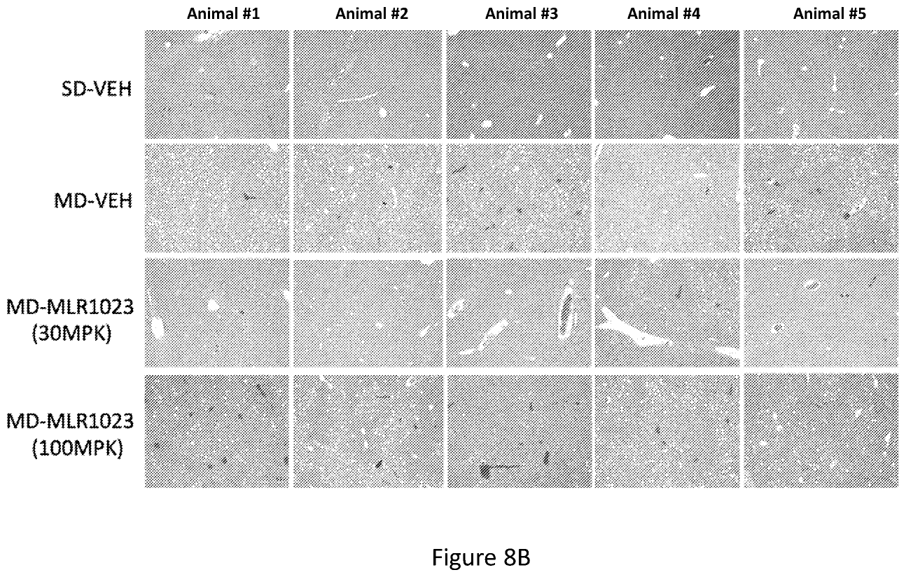

At the end of the study, the liver tissues were collected for histology assessment for NAFLD activity score (NAS). Compared to Normal chow-fed mice, MD-fed Vehicle mice demonstrated significant higher TG deposition in liver. Compared to MD-vehicle mice, MLR-1023 (30 mpk) significantly reduced the TG content in liver (see, FIG. 2). Histological evaluation of liver samples revealed a reduced amount of hepatocellular ballooning in mice treated with MLR-1023 (see, FIGS. 7, 8A, and 8B). Based on the pathology scores, the NAS were also calculated. Compared to Normal chow-fed mice, MD-fed Vehicle mice demonstrated significantly higher NAS in liver (see, FIGS. 6, 8A, and 8B). Compared to MD-vehicle mice, MLR-1023 (30 mpk) significantly reduced NAS. Data are mean±SEM and analyzed by Unpaired T-tests as applicable. MLR-1023 demonstrated activity in liver tissue, independent of its action as an insulin sensitizer by reducing liver triglyceride levels, with a concomitant reduction in liver ballooning as steatosis which are definitive conditions of NASH.

### Example 3: Liver Fibrosis Animal Model

Liver fibrosis is a wound healing response to acute or chronic injury that results in the excessive deposition of extracellular matrix proteins, i.e., scar tissue. Advanced liver fibrosis results in cirrhosis, liver failure, and portal hypertension. In mice, liver fibrosis induced by carbon tetrachloride (CCl₄) resembles important properties of human liver fibrosis including inflammation, regeneration, and fiber formation. This model is commonly used to examine acute liver injury, advanced fibrosis, and fibrosis reversal.

In the following study, 6 to 7 week old male BALB/c mice (25-30 g) were randomized based on body weight after 7-day of CCl₄ injections (3 times). Mice were fed standard rodent chow and water ad libitum. The mice were divided into 4 groups, with 12 mice per group. The mice were administered a dose volume of 10 mL/kg (MLR-1023), 2.5 mL/kg (CCl₄), each in a formulation of 20% HBPCD/saline and corn oil intraperitoneally (i.p.). The dose level of MLR-1023 was 30 and 100 mg per kg, and the dose level of CCl₄ was 5%. The dose frequency of MLR-1023 was once daily (QD), and the dose frequency of CCl₄ was 3 times a week (Monday, Wednesday, and Friday). The study duration was 28 days, with MLR-1023 treatment started from day 8 to day 28. The study design and drug treatment phase is set forth in Table 1.

TABLE 1
Study Design-Drug Treatment Phase

| | | | | Dose | |
|---|---|---|---|---|---|
| Group | | Group | Days of | and | Evaluations/ |
| # | Treatment | Size | Dosing: | Route | Endpoints |
| 1 | Sham (Corn Oil) + | 10 | 21 | IP | Terminal blood/ |
| | Vehicle | | (Vehicle | | serum collection |
| 2 | CCl₄ + Vehicle | 12 | or Test | | and liver |
| 3 | CCl₄ + MLR- | | compound | | collection; |
| | 1023(30 mpk) | | will be | | Liver |
| 4 | CCl₄ + MLR- | | dosed after | | Hydroxyproline |
| | 1023(100 mpk) | | 3^{rd} CCl₄ | | content; Liver |
| | | | injection | | histopathology |
| | | | i.e. Day 8 | | (H&E and PSR; |
| | | | | | n = 5-7/group); |
| | | | | | Terminal serum |
| | | | | | ALT and AST; |

CCl₄ was obtained from Sigma-Aldrich (St. Louis, Mo.). A commercial kit was used for analyzing liver hydroxyproline content (BioVision, Inc.; Milpitas, Calif.). ALT and AST kits were purchased from BIOO Science (Austin, Tex.). All other reagents used in the study were of analytical grade. Male BALB/c mice were obtained from Charles River Laboratories (Wilmington, Mass.). On the last day of the study all mice were sacrificed by cervical dislocation. Blood/serum samples were collected for the assays of liver enzymes (ALT and AST). Livers were rapidly dissected and part of each liver was cut and fixed in formaldehyde saline (4%) solution for histological analysis; the remainder of the liver was snap frozen in liquid nitrogen, and stored at −70° C. until use.

### Induction of Liver Fibrosis by CCl₄:

Starting on Day 1, animals were administered i.p. with 5% CCl₄ or corn oil (vehicle for CCl₄ solution) three days per week (Monday, Wednesday, and Friday) for 4 weeks. CCl₄ was formulated as a 20% solution with the dose volume of 2.5 mL/kg, and was freshly formulated on a weekly basis. Animals were weighed weekly. Test compounds and vehicle administrations were performed from Day 8 to Day 28. At the end of study, blood was drawn through retro-orbital punctures for serum collection. The whole liver was removed. One lobe was placed in a tube containing 10% formalin for histopathology, the remainder of the liver lobes were collected and snap frozen for further hydroxyproline assay. Homogenized liver samples were evaluated for hydroxyproline content to assess hepatic collagen levels.

### Biochemical Assays:

Serum was separated by centrifugation at 4° C. and analyzed immediately or stored at −70° C. Serum ALT and AST levels were determined by colorimetric method and using procedures described by the kit manufacturers. Hepatic hydroxyproline content was measured by colorimetric method and expressed as μg/mg wet liver weight.

### Histology Analysis:

Livers were removed at necropsy and fixed in 10% buffered formalin. Sections of liver were processed routinely, sectioned at approximately 4 microns, and stained with hematoxylin and eosin (H&E) or picrosirius red (PSR). Glass slides were evaluated using light microscopy by a board-certified veterinary/toxicology pathologist in a blind manner. Two sections from each animal were examined. Five animals from each group were randomly chosen for histology analysis. The severity of histologic findings was scored using the Society of Toxicologic Pathology best practices (1=minimal, 2=mild, 3=moderate, 4=marked, 5=Severe). Fibrosis value is corresponding to the fraction of centrilobular areas affected by fibrosis (1=about <10%, 2=about 11-30%, 3=about 39-50%, 4=about 59-70%, 5=about 71-100%).

Liver fibrosis is characterized by increased numbers of fibroblasts and collagen fibers in liver sinusoids that partially, or completely, bridged centrilobular areas. PSR staining severity generally correlated with the mean severity of fibrosis. Overall increased collagen deposition and bridging fibrosis are identified with PSR stain. Subacute centrilobular inflammation which is reflected by infiltration of neutrophils and mononuclear cells in centrilobular zones with H&E staining; mineralization and hepatocellular necrosis characterized by hypereosinophilia and loss of cytoplasmic detail with retained architecture are also scored with H&E staining. Data scores are mean±SEM and analyzed by Unpaired T-tests as applicable. Slides were scored according to the following detailed criteria:

Fibrosis:
1)<10% of centrilobular areas affected by fibrosis
2) 11-30% of centrilobular areas affected by fibrosis
3) 31-50% of centrilobular areas affected by fibrosis
4) 51-70% of centrilobular areas affected by fibrosis
5) 71-100% of centrilobular areas affected by fibrosis

Mineralization/Necrosis:
1) rare, scattered foci of mineralization or hypereosinophilic cells with loss of cytoplasmic detail
2) more numerous foci affecting up to 25% of the centrilobular areas
3) foci of mineralization or necrosis affecting up to 50% of the centrilobular areas 4) foci of mineralization or necrosis affecting up to 75% of the centrilobular areas 5) foci of mineralization or necrosis affecting >75% of the centrilobular areas

Centrilobular Inflammation:
1) minimal—rare inflammatory cells identified
2) mild—small foci of inflammatory cells randomly scattered
3) moderate—more regular observation of inflammatory foci
4) marked—inflammation affecting the preponderance of affected areas
5) severe—diffuse inflammation affecting large portions of the affected liver

Data are expressed as the average±SEM at each week and analyzed by Unpaired T-tests as applicable using Prism® software (GraphPad Software, Inc., Las Jolla, Calif.). A p-value of less than 0.05 indicates a significant difference between treatment groups.

### Serum ALT and AST

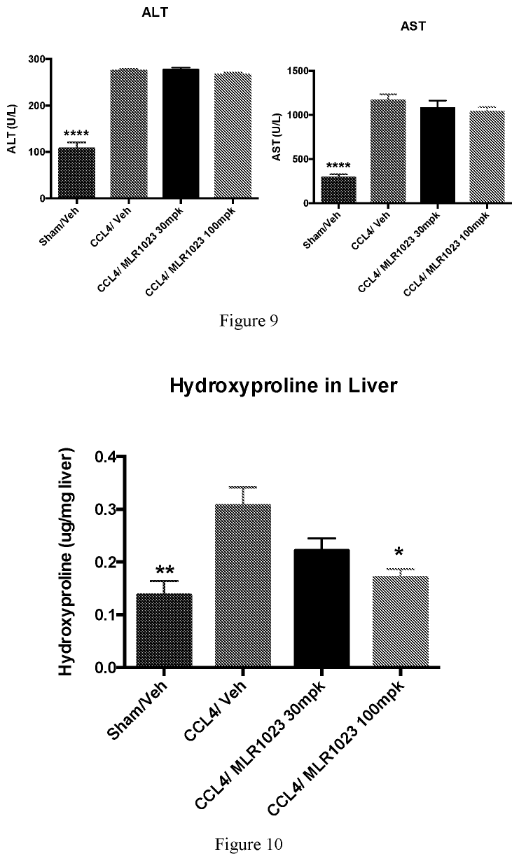

FIG. 9 shows terminal blood liver enzymes changes due to the compound treatments. Serum samples were collected and measured for liver enzymes levels including ALT and AST at the end of the study. Compared to Sham-Vehicle, CCl₄-Vehicle mice had significantly higher ALT and AST levels. Compared to CCl₄-vehicle mice, MLR-1023 treatments did not significantly affect ALT levels, but trended towards improving the AST levels in a dose-dependent manner. Data are mean SEM and analyzed by unpaired T-tests as applicable (****p<0.001 vs. CCl₄-Vehicle). The results indicate that MLR-1023 had mild improvement on CCl₄-induced increase of liver enzymes, especially for AST.

### Hydroxyproline in Liver

FIG. 10 shows hydroxyproline content changes due to the compound treatments. Hydroxyproline content in liver reflects hepatic collagen deposition and further indicates the severity of liver fibrosis. Homogenized liver samples were evaluated for hydroxyproline content. Compared to Sham-Vehicle, CCl₄-Vehicle mice had significantly higher hepatic hydroxyproline content, suggesting increased collagen deposition. Compared to CCl₄-vehicle mice, MLR-1023 treatments significantly reduced hepatic hydroxyproline levels in a dose-dependent manner. Data are mean SEM and analyzed by unpaired T-tests as applicable (*p<0.05; **p<0.01 vs. CCl₄-Vehicle). The results indicate that MLR-1023 significantly reduced CCl₄-induced collagen deposition.

### Histology Analysis:

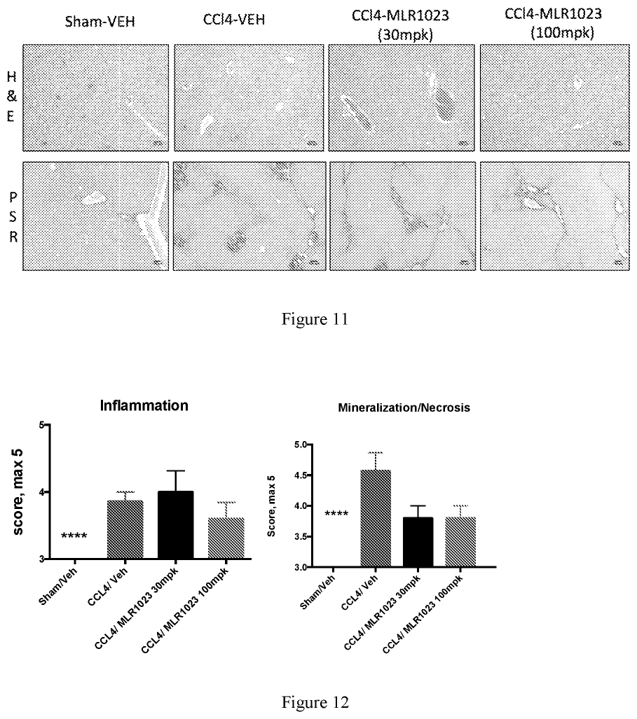

FIG. 11 shows hepatic histological changes due to the compound treatments. At the end of the study, the liver tissues were collected for histology assessment for hepatic lesions (H&E staining) and fibrosis (PSR staining).

### Pathologist's General Assessment:

Animals injected with corn oil (vehicle for CCl₄) and vehicle had normal livers. CCl₄ injections caused centrilobular degeneration and necrosis. In CCl₄-vehicle treated animals, the change was characterized by inflammation, hepatocellular degeneration (hypereosinophilia, loss of detail, nuclear pyknosis and karryorhexis, etc) and multifocal mineralization. Mineralization was more prominent in some animals than others. There was fibrosis associated with these lesions, visible on PSR stained slides. In many animals, fibrosis bridged adjacent centrilobular regions. Treatment with MLR1023 appeared to have a dose-related effect on both necrosis and fibrosis. With both treatments, areas of necrosis were still present but were smaller and did not bridge between centrilobular zones as often. Inflammation was still present with the lesions but necrosis and fibrosis appeared reduced.

FIG. 12 shows histological scoring of inflammation and mineralization/necrosis. Compared to Sham-Vehicle, CCl₄-Vehicle mice had significantly increased scores in inflammation, hepatocellular degeneration-necrosis, multifocal mineralization and fibrosis percentage. Compared to CCl₄-vehicle mice, MLR-1023 treatments trended towards reducing the scores of hepatic necrosis/mineralization. MLR1023 (100 mg per kg) treatment reduced the scores of hepatic inflammation. Data are mean SEM and analyzed by unpaired T-tests as applicable (****p<0.001 vs. CCl₄-Vehicle). The results indicate that MLR-1023 improves CCl₄-induced hepatic lesions and fibrosis.

In summary, the results indicate the following in CCl₄-induced liver fibrosis mouse model with a 21-day treatment with MLR-1023: 1) MLR-1023 displayed mild effects on CCl₄-induced increase of liver enzymes, especially for AST; 2) 100 mg per kg of MLR-1023 significantly reduced CCl₄-induced collagen deposition; 3) MLR-1023 treatments reduced CCl₄-induced hepatic necrosis/mineralization; and 4) MLR-1023 (100 mg per kg) treatment reduced CCl₄-induced hepatic inflammation and fibrosis percentage.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

What is claimed is:

1. A method of treating non-alcoholic steatohepatisis (NASH), non-alcoholic fatty liver disease (NAFLD), fatty acid liver disease (FALD), alcoholic liver disease, and/or liver fibrosis in a mammal in need thereof, comprising administering to the mammal a compound having the formula: 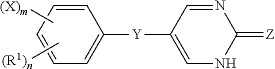
wherein:
R¹ is an alkyl group;
X is a halogen;
Y is O, S, or NH;
Z is O or S; and
n is an integer from 0 to 5 and m is 0 or 1, wherein m+n is less than or equal to 5;
or a pharmaceutically acceptable salt thereof.

2. The method of claim 1, wherein the alkyl group is methyl and n is 1.

3. The method of claim 1 or claim 2, wherein the halogen is chlorine and m is 1.

4. The method of any one of claims 1 to 3, wherein Y is O.

5. The method of any one of claims 1 to 4, wherein Z is O.

6. The method of claim 1, wherein R¹ is methyl, Y is O, Z is O, n is 1, and m is 0.

7. The method of claim 6, wherein R¹ is in the meta position.

8. The method of claim 1, wherein X is chlorine, Y is O, Z is O, n is 0, and m is 1.

9. The method of claim 8, wherein X is in the meta position.

10. The method of claim 1, wherein the lyn kinase activator is of the formula: 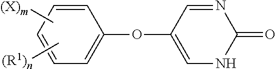
wherein:
R¹ is an alkyl group;
X is a halogen; and
n is an integer from 0 to 5 and m is 0 or 1, wherein m+n is less than or equal to 5;
or a pharmaceutically acceptable salt thereof.

11. The method of claim 10, wherein the alkyl group is methyl and n is 1.

12. The method of claim 10 or claim 11, wherein the halogen is chlorine and m is 1.

13. The method of claim 10, wherein R is methyl, n is 1, and m is 0.

14. The method of claim 13, wherein R is in the meta position.

15. The method of claim 12, wherein X is chlorine, n is 0, and m is 1.

16. The method of claim 15, wherein X is in the meta position.

17. The method of claim 1, wherein the lyn kinase activator is of the formula: 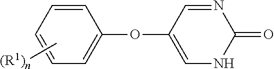
wherein R¹ is an alkyl group and n is an integer from 0 to 5; or a pharmaceutically acceptable salt thereof.

18. The method of claim 17, wherein R is methyl, n is 1.

19. The method of claim 18, wherein R is in the meta position.

20. The method of claim 1, wherein the lyn kinase activator is of the formula: 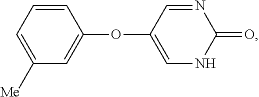 [SMILES: Cc1cccc(Oc2cnc(=O)[nH]c2)c1]
or a pharmaceutically acceptable salt thereof.

21. The method of claim 1, wherein the lyn kinase activator is of the formula: 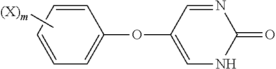
wherein X is a halogen and m is an integer from 0 to 1; or a pharmaceutically acceptable salt thereof.

22. The method of claim 21, wherein X is chloro and m is 1.

23. The method of claim 22, wherein X is in the meta position.

24. The method of claim 1, wherein the lyn kinase activator is of the formula: 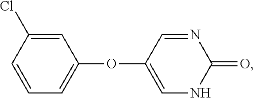 [SMILES: O=c1ncc(Oc2cccc(Cl)c2)c[nH]1]
or a pharmaceutically acceptable salt thereof.

25. The method of claim 1, wherein the lyn kinase activator is of the formula: 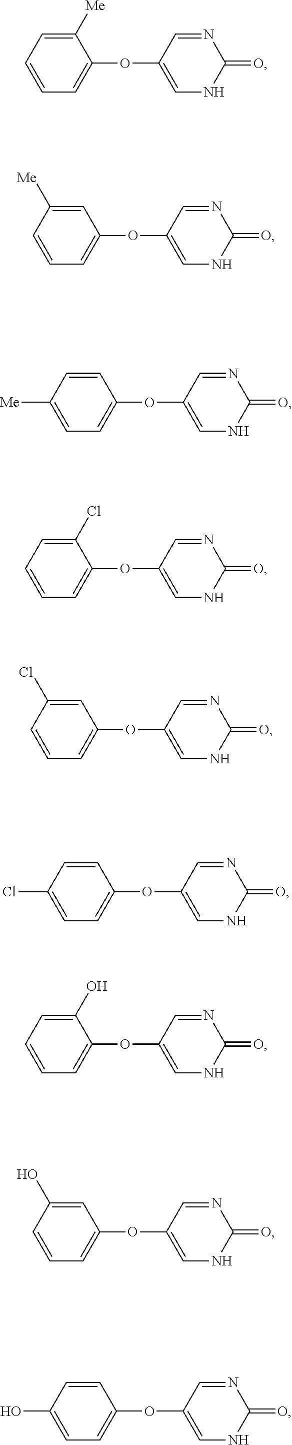 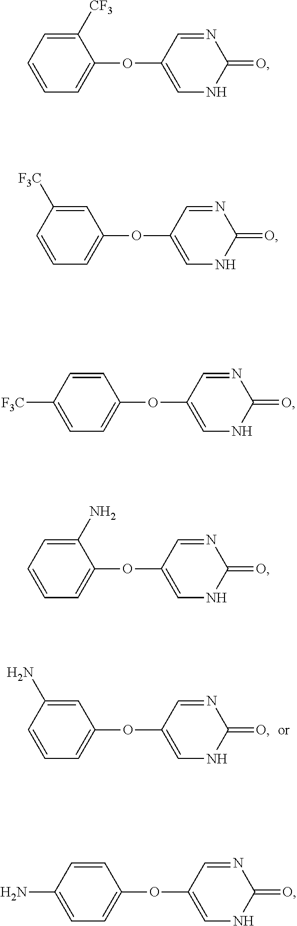 [SMILES: O=c1ncc(Oc2ccccc2C(F)(F)F)c[nH]1 ; O=c1ncc(Oc2cccc(C(F)(F)F)c2)c[nH]1 ; O=c1ncc(Oc2ccc(C(F)(F)F)cc2)c[nH]1 ; Nc1ccccc1Oc1cnc(=O)[nH]c1 ; Nc1cccc(Oc2cnc(=O)[nH]c2)c1 ; Nc1ccc(Oc2cnc(=O)[nH]c2)cc1]
pharmaceutically acceptable salt thereof.

26. A method of treating non-alcoholic steatohepatisis (NASH), non-alcoholic fatty liver disease (NAFLD), fatty acid liver disease (FALD), alcoholic liver disease, and/or liver fibrosis in a mammal in need thereof, comprising administering to the mammal a compound having the formula: 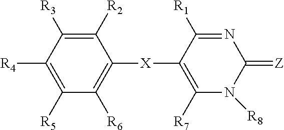
wherein:
each of R₁, R₂, R₃, R₄, R₅, R₆, and R₇ is, independently, a hydrogen, alkoxy, alkyl, alkenyl, alkynyl, aryl, aryloxy, benzyl, cycloalkyl, halogen, heteroaryl, heterocycloalkyl, —CN, —OH, —NO₂, —CF₃, —CO₂H, —CO₂alkyl, or —NH₂;
R₈ is an alkyl or hydrogen;
X is O, S, NH, or N-alkyl; and
Z is O or S; or a pharmaceutically acceptable salt thereof.

27. A method of treating non-alcoholic steatohepatisis (NASH), non-alcoholic fatty liver disease (NAFLD), fatty acid liver disease (FALD), alcoholic liver disease, and/or liver fibrosis in a mammal in need thereof, comprising administering to the mammal a compound having the formula: 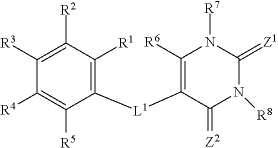
wherein:
each of R₁, R₂, R₃, R₄, and R₅ is, independently, H, halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, NR^{c1}S(O)₂NR^{c1}R^{d1}, S(O)R^{b1}, S(O)NR^{c1}R^{d1}, S(O)₂R^{b1}, NR^{c1}S(O)₂R^{b1}, or S(O)₂NR^{c1}R^{d1}, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4, or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2};
or two adjacent groups of R¹, R², R³, R⁴, and R⁵ can link to form a fused cycloalkyl or fused heterocycloalkyl group, each optionally substituted by 1, 2, or 3 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2};
R⁶ is H, halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, NR^{c1}S(O)₂NR^{c1}R^{d1}, S(O)R^{b1}, S(O)NR^{c1}R^{d1}, S(O)₂R^{b1}, NR^{c1}S(O)₂R^{b1}, or S(O)₂NR^{c1}R^{d1}, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4, or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2};
R⁷ is H, C₁₋₆alkyl, C₁₋₆haloalkyl, C₁₋₆hydroxyalkyl, C(O)R^{b1}, C(O)NR^{c1}R^{d1}, C(O)OR^{a1}, S(O)R^{b1}, S(O)NR^{c1}R^{d1}, S(O)₂R^{b1}, or S(O)₂NR^{c1}R^{d1};
R⁸ is H, C₁₋₆alkyl, C₁₋₆haloalkyl, C₁₋₆hydroxyalkyl, C(O)R^{b1}, C(O)NR^{c1}R^{d1}, C(O)OR^{a1}, S(O)R^{b1}, S(O)NR^{c1}R^{d1}, S(O)₂R^{b1}, or S(O)₂NR^{c1}R^{d1};
R^{a1}, R^{b1}, R^{c1}, and R^{d1} are each, independently, selected from H, C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, and heterocycloalkylalkyl, wherein each of C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, and heterocycloalkylalkyl is optionally substituted with 1, 2, 3, 4, or 5 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy;
or R^{c1} and R^{d1} together with the N atom to which they are attached form a 4-, 5-, 6-, or 7-membered heterocycloalkyl group or heteroaryl group, each optionally substituted with 1, 2, or 3 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy;
R^{a2}, R^{b2}, R^{c2}, and R^{d2} are each, independently, selected from H, C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, and heterocycloalkylalkyl, wherein each of C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, or heterocycloalkylalkyl is optionally substituted with 1, 2, or 3 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy;
or R^{c2} and R^{d2} together with the N atom to which they are attached form a 4-, 5-, 6-, or 7-membered heterocycloalkyl group or heteroaryl group, each optionally substituted with 1, 2, or 3 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy;
Z¹ is O, S, or NR⁹;
R⁹ is H, OH, C₁₋₆alkoxy, C₁₋₆haloalkyl, C₁₋₆hydroxyalkyl, aryloxy, heteroaryloxy, CN, or NO₂;
Z² is O, S, or NR¹⁰;
R¹⁰ is H, OH, C₁₋₆alkoxy, C₁₋₆haloalkyl, C₁₋₆hydroxyalkyl, aryloxy, heteroaryloxy, CN, or NO₂;
L¹ is O, S, or NR¹¹; and
R¹¹ is H, C₁₋₆alkyl, C₁₋₆haloalkyl, C₁₋₆hydroxyalkyl, C(O)R^{b1}, C(O)NR^{c1}R^{d1}, C(O)OR^{a1}, S(O)R^{b1}, S(O)NR^{c1}R^{d1}, S(O)₂R^{b1}, or S(O)₂NR^{c1}R^{d1}; or a pharmaceutically acceptable salt thereof.

28. A method of treating non-alcoholic steatohepatisis (NASH), non-alcoholic fatty liver disease (NAFLD), fatty acid liver disease (FALD), alcoholic liver disease, and/or liver fibrosis in a mammal in need thereof, comprising administering to the mammal a compound having the formula: 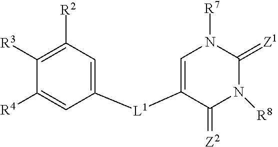
wherein:
R², R³, and R⁴ are each, independently, H, halo, C₁₋₆alkyl, C₁₋₆hydroxyalkyl, or C₁₋₆haloalkyl;
R⁷ is H, C₁₋₆alkyl, C(O)R^{b1}, C(O)NR^{c1}R^{d1}, or C(O)OR^{a1};
R⁸ is H, C₁₋₆alkyl, C(O)R^{b1}, C(O)NR^{c1}R^{d1}, or C(O)OR^{a1},
R^{a1}, R^{b1}, R^{c1}, and R^{d1} are each, independently, selected from H, C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, and heterocycloalkylalkyl, wherein each of C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, and heterocycloalkylalkyl is optionally substituted with 1, 2, 3, 4, or 5 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy;
or R^{c1} and R^{d1} together with the N atom to which they are attached form a 4-, 5-, 6-, or 7-membered heterocycloalkyl group or heteroaryl group, each optionally substituted with 1, 2, or 3 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy;
Z¹ is O or S;
Z² is O or S; and
L¹ is O or S;
or a pharmaceutically acceptable salt thereof.

29. A method of treating non-alcoholic steatohepatisis (NASH), non-alcoholic fatty liver disease (NAFLD), fatty acid liver disease (FALD), alcoholic liver disease, and/or liver fibrosis in a mammal in need thereof, comprising administering to the mammal a compound having the formula: 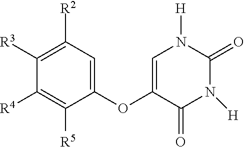
wherein:
R², R³, R⁴, and R⁵ are each, independently, H, F, Cl, CH₃, SCH₃, OCH₃, C(CH₃)₃, CH(CH₃)₂, or C₂H₅; or a pharmaceutically acceptable salt thereof.

30. A method of treating non-alcoholic steatohepatisis (NASH), non-alcoholic fatty liver disease (NAFLD), fatty acid liver disease (FALD), alcoholic liver disease, and/or liver fibrosis in a mammal in need thereof, comprising administering to the mammal a compound having the formula: 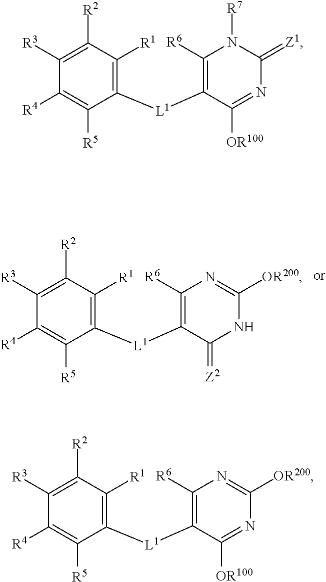
wherein:
each of R₁, R₂, R₃, R₄, and R₅ is, independently, H, halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, NR^{c1}S(O)₂NR^{c1}R^{d1}, S(O)R^{b1}, S(O)NR^{c1}R^{d1}, S(O)₂R^{b1}, NR^{c1}S(O)₂R^{b1}, or S(O)₂NR^{c1}R^{d1} wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4, or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2} NR^{c2}S(O)₂R^{b2}, and S(O)₂NR^{c2}R^{d2};
or two adjacent groups of R¹, R², R³, R⁴, and R⁵ can link to form a fused cycloalkyl or fused heterocycloalkyl group, each optionally substituted by 1, 2, or 3 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2};
R⁶ is H, halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)NR^{c1}R^{d1}, NR^{c1}C(O)OR^{a1}, NR^{c1}S(O)₂NR^{c1}R^{d1}, S(O)R^{b1}, S(O)NR^{c1}R^{d1}, S(O)₂R^{b1}, NR^{c1}S(O)₂R^{b1}, or S(O)₂NR^{c1}R^{d1}, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4, or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2};
R⁷ is H, C₁₋₆alkyl, C₁₋₆haloalkyl, C(O)R^{b1}, C(O)NR^{c1}R^{d1}, C(O)OR^{a1}, S(O)R^{b1}, S(O)NR^{c1}R^{d1}, S(O)₂R^{b1}, or S(O)₂NR^{c1}R^{d1};
R^{a1}, R^{b1}, R^{c1}, and R^{d1} are each, independently, selected from H, C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, and heterocycloalkylalkyl, wherein each of C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, and heterocycloalkylalkyl is optionally substituted with 1, 2, 3, 4, or 5 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy;
or R^{c1} and R^{d1} together with the N atom to which they are attached form a 4-, 5-, 6-, or 7-membered heterocycloalkyl group or heteroaryl group, each optionally substituted with 1, 2, or 3 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy;
R^{a2}, R^{b2}, R^{c2}, and R^{d2} are each, independently, selected from H, C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, and heterocycloalkylalkyl, wherein each of C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, arylalkyl, heteroarylalkyl, cycloalkylalkyl, or heterocycloalkylalkyl is optionally substituted with 1, 2, or 3 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy;
or R^{c2} and R^{d2} together with the N atom to which they are attached form a 4-, 5-, 6-, or 7-membered heterocycloalkyl group or heteroaryl group, each optionally substituted with 1, 2, or 3 substituents independently selected from OH, NO₂, CN, amino, halo, C₁₋₆alkyl, C₁₋₆alkoxy, C₁₋₆haloalkyl, C₁₋₆hydroxyalkyl, and C₁₋₆haloalkoxy;
Z¹ is O, S, or NR⁹;
R⁹ is H, OH, C₁₋₆alkoxy, C₁₋₆haloalkyl, C₁₋₆hydroxyalkyl, aryloxy, heteroaryloxy, CN, or NO₂;
Z² is O, S, or NR¹⁰;
R¹⁰ is H, OH, C₁₋₆alkoxy, C₁₋₆haloalkyl, C₁₋₆hydroxyalkyl, aryloxy, heteroaryloxy, CN, or NO₂;
L¹ is O, S, or NR¹¹;
R¹¹ is H, C₁₋₆alkyl, C₁₋₆haloalkyl, C₁₋₆hydroxyalkyl, C(O)R^{b1}, C(O)NR^{c1}R^{d1}, C(O)OR^{a1}, S(O)R^{b1}, S(O)NR^{c1}R^{d1}, S(O)₂R^{b1}, or S(O)₂NR^{c1}R^{d1};
R¹⁰⁰ is a hydroxyl protecting group, C₁₋₆alkyl, C₂₋₆alkenyl, C₂₋₆alkynyl, C₁₋₆haloalkyl, C₁₋₆hydroxyalkyl, C₃₋₆cycloalkyl, heterocycloalkyl, aryl, heteroaryl, C(O)R^{b1}, C(O)NR^{c1}R^{d1}, C(O)OR^{a1}, S(O)R^{b1}, S(O)NR^{c1}R^{d1}, S(O)₂R^{b1}, S(O)₂NR^{c1}R^{d1}, S(O)₂OR^{e1}, P(O)OR^{f1}OR^{g1}, or Si(R^{h1})₃, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4 or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2};
R²⁰⁰ is a hydroxyl protecting group, C₁₋₆alkyl, C₂₋₆alkenyl, C₂₋₆alkynyl, C₁₋₆haloalkyl, C₁₋₆hydroxyalkyl, C₃₋₆cycloalkyl, heterocycloalkyl, aryl, heteroaryl, C(O)R^{b1}, C(O)NR^{c1}R^{d1}, C(O)OR^{a1}, S(O)R^{b1}, S(O)NR^{c1}R^{d1}, S(O)₂R^{b1}, S(O)₂NR^{c1}R^{d1}, S(O)₂OR^{e1}, P(O)OR^{f1}OR^{g1}, or Si(R^{h1})₃, wherein each of C₁₋₆alkyl, C₂₋₆alkenyl, C₂₋₆alkynyl, C₁₋₆haloalkyl, C₁₋₆hydroxyalkyl, C₃₋₆cycloalkyl, aryl, and heteroaryl, is optionally substituted by 1, 2, 3, 4 or 5 substituents independently selected from halo, C₁₋₆alkyl, C₂₋₆alkenyl, C₂₋₆alkynyl, C₁₋₆haloalkyl, C₁₋₆hydroxyalkyl, C₃₋₆cycloalkyl, aryl, heteroaryl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)NR^{c2}R^{d2}, NR^{c2}C(O)OR^{a2}, NR^{c2}S(O)NR^{c2}R^{d2}, S(O)R^{b2}, S(O)NR^{c2}R^{d2}, S(O)₂R^{b2}, NR^{c2}S(O)₂R^{b2}, and S(O)₂NR^{c2}R^{d2};
each R^{e1} is, independently, H, C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, cycloalkylalkyl, heterocycloalkylalkyl, arylalkyl, or heteroarylalkyl;
each R^{f1} is, independently, H, C₁₋₆alkyl, C₁₋₆haloalkyl, C₁₋₆hydroxyalkyl, C₂₋₆alkenyl, (C₁₋₆alkoxy)-C₁₋₆alkyl, C₂₋₆alkynyl, aryl, cycloalkyl, heteroaryl, heterocycloalkyl, arylalkyl, cycloalkylalkyl, heteroarylalkyl, or heterocycloalkylalkyl;
each R^{g1} is, independently, H, C₁₋₆alkyl, C₁₋₆haloalkyl, C₁₋₆hydroxyalkyl, C₂₋₆alkenyl, C₂₋₆alkynyl, aryl, cycloalkyl, heteroaryl, or heterocycloalkyl; and
each R^{h1} is, independently, H, C₁₋₆alkyl, C₁₋₆haloalkyl, C₁₋₆hydroxyalkyl, C₂₋₆alkenyl, C₂₋₆alkynyl, aryl, cycloalkyl, heteroaryl, heterocycloalkyl, cycloalkylalkyl, heterocycloalkylalkyl, arylalkyl, or heteroarylalkyl; or a pharmaceutically acceptable salt thereof.

31. The method of any one of claims 1 to 56 further comprising administering to the mammal any one or more of a statin, a PPAR agonist, a bile-acid-binding resin, niacin, nicotinic acid, a RXR agonist, an anti-obesity drug, a hormone, a tyrophostine, a sulfonylurea-based drug, a biguanide, an α-glucosidase inhibitor, an apo A-I agonist, a cardiovascular drug, a chemotherapeutic agent, an FXR agonist, a PPARα agonist, a GLP-1 agonist, a PPARu/6 dual agonist, an ACC inhibitor, a growth factor, a CCR2/5 blocker, and a anti-liver disease therapeutic agent.
